(19) United States
(12) Patent Application Publication
(10) Pub. No.: US 2010/0113326 A1
(43) Pub. Date: May 6, 2010

(54) DIMERIC IAP INHIBITORS

(75) Inventor: Stephen M. Condon, Glenmoore, PA (US)

Correspondence Address: BANNER & WITCOFF, LTD., 1100 13th STREET, N.W., SUITE 1200, WASHINGTON, DC, 20005-4051, US

(73) Assignee: TETRALOGIC PHARMACEUTICALS CORPORATION, Malvern, PA (US)

(21) Appl. No.: 12/374,724

(22) PCT Filed: Jul. 24, 2007
(86) PCT No.: PCT/US07/74186

§ 371 Date: Dec. 2, 2009

Related U.S. Application Data
(60) Provisional application No. 60/820,172, filed on Jul. 24, 2006.

(51) Int. Cl. A61K 38/02; C07K 2/00; A61P 35/00; A61P 37/00

(52) U.S. Cl. 514/2; 530/300

(57) ABSTRACT

Compounds made up of a homodimer or heterodimer having monomeric units of formula (I): wherein: each A is, independently, NR₁R₂, or N⁺R₁R₂R₃; each B is, independently, optionally substituted alkyl, alkenyl, or alkynyl, wherein one or more hydrogens are optionally replaced by fluorine; each U is, independently, CONH, C(O)O, C(S)O, C(S)NH, C(NH)NH, (CH₂)₁₋₅, or the inverse, thereof, wherein one or more carbons are optionally replaced by a heteroatom selected from O, S, and N; each V and W is, independently, (CH₂)₁₋₅, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅; each X is, independently, optionally substituted alkyl, alkenyl, alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄. SR₄, or NR₄R₅; each Y is, independently, CH or N; each Z is, independently, CH₂, C═O, C═S, CHSR, CHOR, or CHNR, compositions, and methods of using such compounds to modulate apoptosis including IAP antagonists are provided herein. Compositions including mimetics of the invention and, optionally, secondary agents, may be used to treat proliferative disorders such as, cancer and autoimmune diseases.

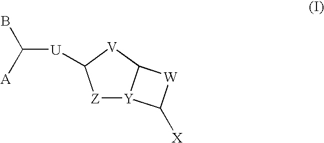

### CROSS-REFERENCE TO RELATED APPLICATIONS

This application claims priority to and benefit of U.S. Provisional Application No. 60/820,172 entitled “Dimeric IAP Inhibitors” filed on Jul. 24, 2006; the entire contents of which is hereby incorporated by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

The invention presented herein provides compositions and methods for modulation of apoptotic signaling pathways.

2. Description of Related Art

Apoptosis plays a central role in the development and homeostasis of all multi-cellular organisms. Alterations in apoptotic pathways have been implicated in many types of human pathologies, including developmental disorders, cancer, autoimmune diseases, and neurodegenerative disorders. One mode of action of chemotherapeutic drugs is cell death via apoptosis.

Apoptosis is conserved across species and executed primarily by activated caspases, a family of cysteine proteases that cleave their substrates specifically at aspartate residues. Caspases are produced in cells as catalytically inactive zymogens (procaspases) that are activated by proteolytic processing during the intitation of apoptosis. Once activated, effector caspases proteolytically activate a broad spectrum of cellular targets ultimately leading to cell death.

In mammalian cells activation of the caspases is achieved through at least two independent mechanisms which are initiated by distinct caspases, but result in the activation of common executioner (effector) caspases. The ‘intrinsic pathway’ is activated by cytochrome c which is released from mitochondria within the cell when apoptosis is initiated. The ‘extrinsic pathway’ is activated via activation of a death receptor located on the cell membrane. During extrinsic activation, death receptors, such as, Fas (CD-95/Apo1) and TNF-R1, as well as other members of the TNF group of cytokine receptors, are activated by their corresponding ligands. Fas ligand (FasL/CD-95L) and TNF-alpha or Apo2 ligand/TNF-related apoptosis inducing ligand (Apo2L/TRAIL), respectfully. Binding of procaspase-8 to an activated death receptor induces cleavage and removal of inhibitory domain of procaspase-8 releasing it from the receptor and allowing it to activate effector caspases-3, -6, and -7. The result is the proteolytic cleavage of cellular targets by the effector caspases and the induction of apoptosis.

In normal cells that have not received an apoptotic stimulus, most caspases remain inactive. Aberrantly activatation of caspases is inhibited by a family of evolutionarily conserved proteins called IAPs (inhibitors of apoptosis proteins). IAPs have been described in organisms ranging from Drosophila to Humans. All mammalian IAPs identified to date, including, for example, XIAP, cIAP-1, cIAP-2, ML-IAP, NAIP, Bruce, and survivin exhibit anti-apoptotic activity in cell culture.

IAPs were originally discovered in Baculovirus by their ability to substitute for P35, an anti-apoptotic protein. Generally, IAPs are made up of one to three Baculovirus IAP repeat (BIR) domains, and must also possess a carboxyl-terminal RING finger motif. The BIR domain itself includes a zinc binding domain of about 70 residues made up of 4 alpha-helices and 3 beta strands. The BIR domain itself is believed to inhibit apoptosis by interacting with the procaspase and inhibiting proteolytic activation of the procaspase. IAPs are also known to be overexpressed in many human cancers. For example, XIAP is ubiquitously expressed in most adult and fetal tissues. However, overexpression of XIAP in tumor cells has been demonstrated to confer protection against a variety of apoptotic stimuli and promote resistance to chemotherapy. Consistent with this, a strong correlation between XIAP protein levels and survival of patients with acute myelogenous leukemia has been demonstrated. Down-regulation of XIAP expression by antisense oligonucleotides has been shown to sensitize tumor cells to a wide range of pro-apoptotic agents, both in vitro and in vivo.

Smac/DIABLO-derived peptides have also been demonstrated to sensitize tumor cell lines to pro-apoptotic drugs. In non-tumorigenic cells signaled to undergo apoptosis, IAP-mediated inhibition of apoptosis must be eliminated, which is accomplished, at least in part, by Smac (second mitochondrial activator of caspases). Smac, or DIABLO, is synthesized in the cytoplasm as a 239 amino acid precursor protein, of which the N-terminal 55 residues serve as the mitochondria targeting sequence that is removed after import to the mitochondria. Mature Smac, containing 184 amino acids, accumulates in the inter-membrane space of the mitochondria where it has been shown to behave as an oligomer. When apoptosis is induced, Smac is released from the mitochondria into the cytosol together with cytochrome c where it binds to IAPs eliminating the inhibitory effect of IAPs on protealysis of procaspases and enabling caspase activation. At the same time, cytochrome c induces multimerization of Apaf-1 to activate procaspase-9 and procaspase-3.

Smac interacts with essentially all IAPs identified to date including XIAP, c-IAP1, c-IAP2, ML-IAP, Bruce and survivin and may be a master regulator of apoptosis in mammals. X-ray crystallography has shown that the first four amino acids (AVPI) of mature Smac bind to a portion of IAPs and this binding is thought to be essential for blocking the anti-apoptotic effects of IAPs. Therefore, Smac and various fragments of Smac, including AVPI peptides, have been proposed for use as targets for identification of therapeutic agents.

The basic biology of IAP antagonists, such as Smac, suggests that these proteins may complement or synergize other chemotherapeutic/anti-neoplastic agents and/or radiation. Chemotherapeutic/anti-neoplastic agents and radiation would be expected to induce apoptosis as a result of DNA damage and/or the disruption of cellular metabolism.

### BRIEF SUMMARY OF THE INVENTION

Various embodiments of the invention include compounds made up of a homodimer or heterodimer having monomeric units of formula (I):

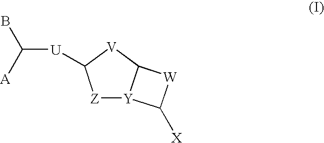

wherein:

each A is, independently, NR₁R₂, or N⁺R₁R₂R₃;

each R₁, R₂, and R₃ is, independently, hydrogen or optionally substituted alkyl, alkenyl, or alkynyl, wherein one or more carbons are optionally replaced by C═O, C═S, or a heteroatom selected from O, S, and N and wherein one or more hydrogens are optionally replaced by fluorine, a branched or unbranched alkyl or cycloalkyl, an optionally substituted aryl, arylalkyl, heteroaryl, heteroarylalkyl, OR₄, SR₄, or NR₄R₅;

each R₄ and R₅ are, independently, hydrogen or optionally substituted alkyl, alkenyl, or alkynyl, wherein one or more carbons are optionally replaced by a heteroatom selected from O, S, and N, or optionally substituted aryl, arylalkyl, heteroaryl, or heteroarylalkyl; or any two of R₁, R₂, and R₃ taken together with the nitrogen to which they are attached to form a heterocyclic group, in which one or more carbon atoms are optionally replaced by C═O, C═S, or a heteroatom selected from O, S, and N, with the proviso that the heteroatom is separated from the nitrogen atom by at least two carbons;

each B is, independently, optionally substituted alkyl, alkenyl, or alkynyl, wherein one or more hydrogens are optionally replaced by fluorine;

each U is, independently, CONH, C(O)O, C(S)O, C(S)NH, C(NH)NH, (CH₂)₁₋₅, or the inverse, thereof, wherein one or more carbons are optionally replaced by a heteroatom selected from O, S, and N;

each V and W is, independently, (CH₂)₁₋₅, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;

each X is, independently, optionally substituted alkyl, alkenyl, alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;

each Y is, independently, CH or N;

each Z is, independently, CH₂, C═O, C═S, CHSR, CHOR, or CHNR; and

each R is, independently, hydrogen or optionally substituted alkyl, alkenyl, or alkynyl; or

a pharmaceutically acceptable salt thereof.

In some embodiments, the compounds of the invention may be of formula (II):

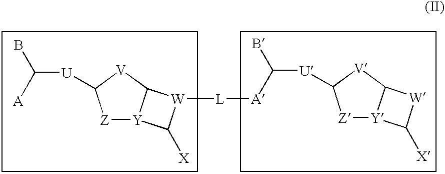

wherein:

A and A′ are each, independently, NR₁R₂ or N⁺ R₁R₂R₃;

R₁, R₂, and R₃ are each, independently, hydrogen or optionally substituted alkyl, alkenyl, or alkynyl, wherein one or more carbons are optionally replaced by C═O, C═S, or a heteroatom selected from O, S, and N, and one or more hydrogens are optionally replaced by fluorine, a branched or unbranched alkyl or cycloalkyl, an optionally substituted aryl, arylalkyl, heteroaryl, heteroarylalkyl, OR₄, SR₄, or NR₄R₅;

R₄ and R₅ are each, independently, hydrogen or optionally substituted alkyl, alkenyl, or alkynyl, in which one or more carbons are optionally replaced by a heteroatom selected from O, S, and N, or optionally substituted aryl, arylalkyl, heteroaryl, or heteroarylalkyl; or any two of R₁, R₂, and R₃ taken together with the nitrogen to which they are attached to form a heterocyclic group, wherein one or more carbon atoms are optionally replaced by C═O, C═S, or a heteroatom selected from O, S, and N, with the proviso that the heteroatom is separated from the nitrogen atom by at least two carbons;

B and B′ are each, independently, optionally substituted alkyl, alkenyl, or alkynyl, wherein one or more hydrogens are optionally replaced by fluorine;

U and U′ are each, independently, CONH, C(O)O, C(S)O, C(S)NH, C(NH)NH, (CH₂)₁₋₅, or the inverse wherein one or more carbons are optionally replaced by a heteroatom selected from O, S, and N;

V, V′, W and W′ are each, independently, (CH₂)₁₋₅, wherein one or more carbons are optionally replaced by C═O, C═S, or a heteroatom selected from O, S, and N, and one or more hydrogens in CH₂ groups are optionally replaced by a branched or unbranched alkyl or cycloalkyl, an optionally substituted aryl, arylalkyl, heteroaryl, heteroarylalkyl, OR₄, SR₄, or NR₄R₅;

X and X′ are each, independently, optionally substituted C₁₋₁₈alkyl, C₂₋₁₈-alkenyl, or C₂₋₁₈-alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;

Y and Y′ are each, independently, CH or N;

Z and Z′ are each, independently, CH₂, C═O, C═S, CHSR, CHOR, or CHNR;

each R is, independently, hydrogen, or optionally substituted alkyl, alkenyl, or alkynyl; and

L is one or more bonds or one or more linkers covalently linking one or more of positions V, W, and X, with V′, W′, and X′; or

a pharmaceutically acceptable salt thereof.

In other embodiments, L may covalently link two identical monomeric units or L may covalently link two non-identical monomeric units. In various embodiments, L may be selected from alkylene, alkenylene, alkynylene, cycloalkylene, cycloalkylalkylene, aryl, arylalkylene, arylalkylalkylene, and heterocycloalkylene, heterocycloalkylalkylene, heteroaryl and heteroarylalkylene where one or more carbon atoms are optionally replaced with N, O, or S, optionally-substituted alkylene, alkenylene, alkynylene cycloalkylene, cycloalkylalkylene, heterocycloalkylene, heterocycloalkylalkylene, aryl, arylalkylene, arylalkylalkylene, and heteroaryl and heteroarylalkylene where one or more carbon atoms are optionally replaced with N, O, or S, amino, substituted amino, oxygen atom, sulfide, sulfoxide, sulfone and disulfide, and in various other embodiments, L may be selected from —CH₂CH₂—, —CH₂CH₂CH₂—, —CH═CH—, 1,4-phenyl, 2,5-thiophenyl, —CH(OH)CH(OH)—, —CH₂CH—O—CHCH₂—, and —CH₂C≡CC≡CCH₂—.

In certain embodiments, the compounds of the invention may have a formula selected from a compound of formula (III):

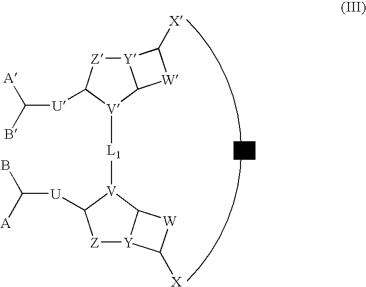

wherein:

A, A′, B, B′, U, U′, Z, Z′, Y, Y′, V, V′, W, W′, X, and X′ are defined as in claim 2; and

L₁ and L₂, independently, link position V with V′ and X with X′; or

a pharmaceutically acceptable salt thereof;
a compound of general formula (IV):

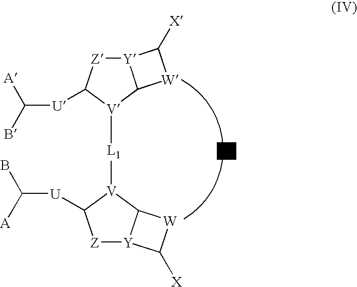

wherein:

A, A′, B, B′, U, U′, Z, Z′, Y, Y′, V, V′, W, W′, X, and X′ are defined as in claim 2; and

L₁ and L₂, independently, link position V with V′ and W with W′; or

a pharmaceutically acceptable salt thereof;
a compound of general formula (V):

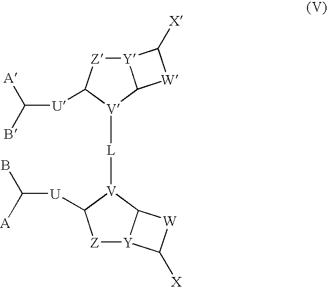

wherein:

A, A′, B, B′, U, U′, Z, Z′, Y, Y′, V, V′, W, W′, X, and X′ are defined as in claim 2; and

L links position V with V′; or

a pharmaceutically acceptable salt thereof;
a compound of general formula (VI):

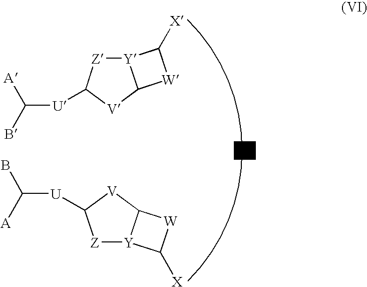

wherein:

A, A′, B, B′, U, U′, Z, Z′, Y, Y′, V, V′, W, W′, X, and X′ are defined as in claim 2; and

L links position X with X′; or

a pharmaceutically acceptable salt thereof; and
a compound of general formula (VII):

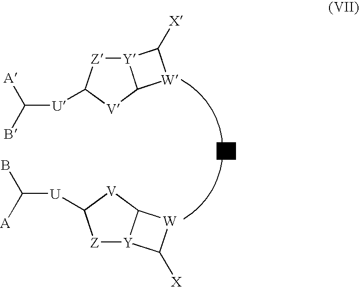

wherein:

A, A′, B, B′, U, U′, Z, Z′, Y, Y′, V, V′, W, W′, X, and X′ are defined as in claim 2; and

L links position W with W′; or

a pharmaceutically acceptable salt thereof.

Various other embodiments of the invention include compounds of a homodimer or heterodimer having monomeric units of formula (VIII):

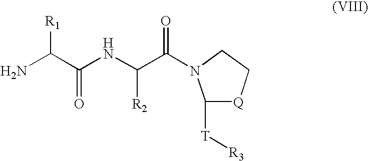

wherein:

each R₁ is, independently, alkyl or haloalkyl;

each R₂ is, independently, branched or unbranched alkyl or cycloalkyl, or substituted or unsubstituted aryl, arylalkyl, heteroaryl, or heteroarylalkyl;

each R₃ is, independently, branched or unbranched alkyl or cycloalkyl, or substituted or unsubstituted aryl, arylalkyl, heteroaryl, or heteroarylalkyl;

each S is, independently, (CH₂)₀₋₃ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and each T is, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHSO₂(CH₂)₀₋₃, (CH₂₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is a branched or unbranched alkyl, cycloalkyl or substituted or unsubstituted aryl, arylalkyl, heteroaryl, or heteroarylalkyl teroaryl; or

a pharmaceutically acceptable salt thereof.

In certain embodiments, the compounds of the invention may be of formula (IX):

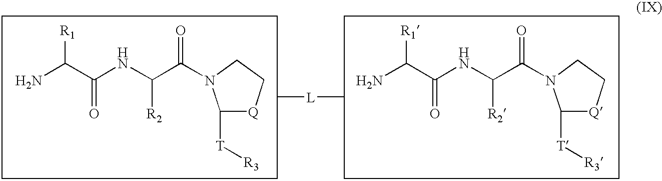

wherein:

R₁ and R₁′ are each, independently, alkyl or haloalkyl;

R₂ and R₂′ are each, independently, branched or unbranched alkyl or cycloalkyl, or substituted or unsubstituted aryl, arylalkyl, heteroaryl, or heteroarylalkyl;

R₃ and R₃′ are each, independently, branched or unbranched alkyl or cycloalkyl, or substituted or unsubstituted aryl, arylalkyl, heteroaryl, or heteroarylalkyl;

Q and Q′ are each, independently, (CH₂)₀₋₃ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

T and T′ are each, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and

L is one or more linkers covalently linking one or more of the positions R₂, R₃, Y, with R₂′, R₃′, Y′; and

pharmaceutically acceptable salts thereof.

In some embodiments, L may covalently link two identical monomeric units or L may covalently link two non-identical monomeric units. In various embodiments, L may be selected from alkylene, alkenylene, alkynylene, cycloalkylene, cycloalkylalkylene, aryl, arylalkylene, arylalkylalkylene, and heterocycloalkylene, heterocycloalkylalkylene, heteroaryl and heteroarylalkylene where one or more carbon atoms are optionally replaced with N, O, or S, optionally-substituted alkylene, alkenylene, alkynylene cycloalkylene, cycloalkylalkylene, heterocycloalkylene, heterocycloalkylalkylene, aryl, aryl alkylene, arylalkylalkylene, and heteroaryl and heteroarylalkylene where one or more carbon atoms are optionally replaced with N, O, or S, amino, substituted amino, oxygen atom, sulfide, sulfoxide, sulfone and disulfide, and in various other embodiments, L may be selected from —CH₂CH₂—, —CH₂CH₂CH₂—, —CH═CH—, 1,4-phenyl, 2,5-thiophenyl, —CH(OH)CH(OH)—, —CH₂CH—O—CHCH₂—, and —CH₂C≡CC≡CCH₂—.

In certain embodiments, the compounds of the invention may be selected from a compound of formula (X):

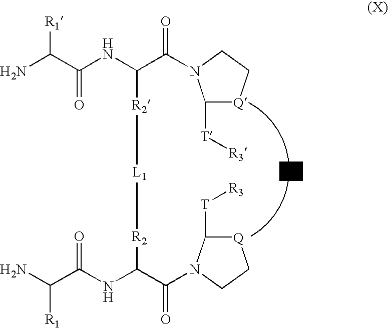

wherein:

R₁, R₁′, R₂, R₂′, R₃, R₃′, Q, Q′, T, and T′ are defined as in claim 11; an

L₁ and L₂, independently, link position R₂ with R₂′ and Q with Q′; or

a pharmaceutically acceptable salt thereof;
a compound of general formula (XI):

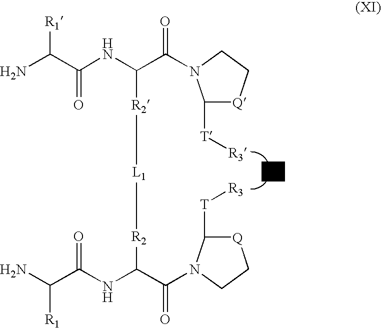

wherein:

R₁, R₁′, R₂, R₂′, R₃, R₃′, Q, Q′, T, and T′ are defined as in claim 11; and

L₁ and L₂, independently, link position R₂ with R₂′ and R₃ with R₃′; or

a pharmaceutically acceptable salt thereof;
a compound of general formula (XII):

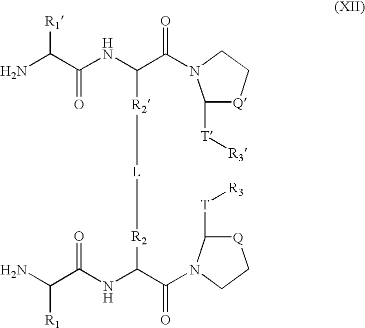

wherein:

R₁, R₁′, R₂, R₂′, R₃, R₃′, Q, Q′, T, and T′ are defined as in claim 11; and

L covalently links position R₂ with R₂′; or

a pharmaceutically acceptable salt thereof;
a compound of general formula (XIII):

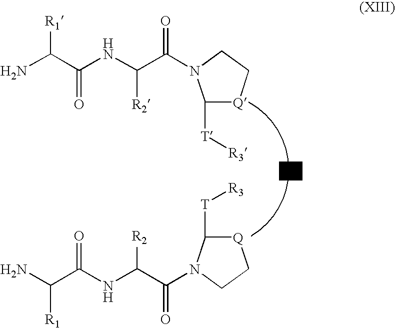

wherein:

R₁, R₁′, R₂, R₂′, R₃, R₃′, Q, Q′, Z, and Z′ are defined as in claim 11; and

L covalently links position Q with Q′; or

a pharmaceutically acceptable salt thereof; and
a compound of general formula (XIV):

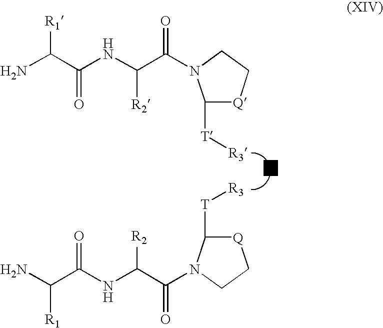

wherein:

R₁, R₁′, R₂, R₂′, R₃, R₃′, Q, Q′, T, and T′ are defined as in claim 11; and

L covalently links position R₃ with R₃′; or

a pharmaceutically acceptable salt thereof.

In further embodiments, the compound of the invention may include a homodimer or heterodimer having monomeric units of general formula (XV):

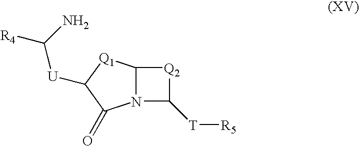

wherein:

each R₄ is, independently, alkyl or haloalkyl;

each R₅ is, independently, branched or unbranched alkyl or cycloalkyl, or substituted or unsubstituted aryl, arylalkyl, heteroaryl, or heteroarylalkyl;

each U is, independently, CONH, CH₂O, CH₂NH, CH₂S, or (CH₂)₁₋₃;

each Q is, independently, (CH₂)₁₋₅ wherein one or more carbons can be replaced by one or more heteroatoms selected from O, S, and N; and one or more hydrogens in CH₂ groups can be replaced by a branched or unbranched alkyl or cycloalkyl, or substituted or unsubstituted aryl, arylalkyl, heteroaryl, or heteroarylalkyl;

each Q₂ is, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

each T is, independently. CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; or

a pharmaceutically acceptable salt thereof.

In still other embodiments, the compounds of the invention may be of formula (XVI):

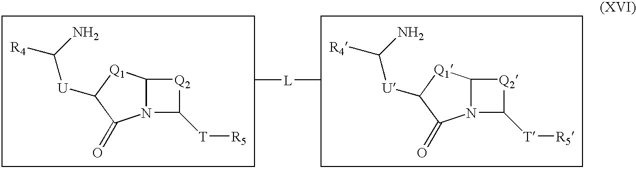

wherein:

R₄ and R₄′ are each, independently, alkyl or haloalkyl;

R₅ and R₅′ are each, independently, branched or unbranched alkyl or cycloalkyl, or substituted or unsubstituted aryl, arylalkyl, heteroaryl, or heteroarylalkyl;

U and U′ are each, independently, CONH, CH₂O, CH₂NH, CH₂S, or (CH₂)₁₋₃;

Q₁ and Q₁′ are each, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

Q₂ and Q₂′ are each, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

T and T′ are each, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and

L is one or more linkers covalently linking one or more of the positions Y₁, Y₂, R₂, with Y₁′, Y₂′, R₂′; or

a pharmaceutically acceptable salt thereof

In some embodiments, L may covalently link two identical monomeric units or L covalently links two non-identical monomeric units. In various embodiments, L may be selected from alkylene, alkenylene, alkynylene, cycloalkylene, cycloalkylalkylene, aryl, arylalkylene, arylalkylalkylene, and heterocycloalkylene, heterocycloalkylalkylene, heteroaryl and heteroarylalkylene where one or more carbon atoms are optionally replaced with N, O, or S, optionally-substituted alkylene, alkenylene, alkynylene cycloalkylene, cycloalkylalkylene, heterocycloalkylene, heterocycloalkylalkylene, aryl, arylalkylene, arylalkylalkylene, heteroaryl, and heteroarylalkylene where one or more carbon atoms are, optionally, replaced with N, O, or S, amino, substituted amino, oxygen atom, sulfide, sulfoxide, sulfone, and disulfide, and in various other embodiments, L may be selected from —CH₂CH—, —CH₂CH₂CH₂—, —CH═CH—, 1,4-phenyl, 2,5-thiophenyl, —CH(OH)CH(OH)—, —CH₂CH—O—CHCH₂—, and —CH₂C═CC≡CCH₂—.

In further embodiments, the compounds of the invention may have a formula selected from a compound of formula (XVII):

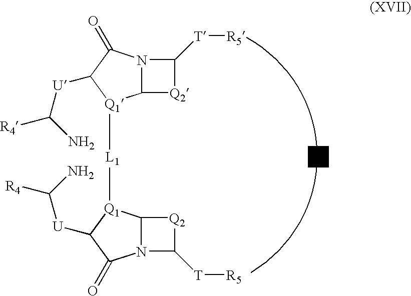

wherein:

R₄, R₄′, U, U′, Q₁, Q₁′, Q₂, Q₂′, T, T′, R₅, and R₅′ are defined as in claim 14; and

L₁ and L₂, independently, link position Q with Q₁′ and R₅ with R₅′; or

a pharmaceutically acceptable salt thereof;
a compound of general formula (XVIII):

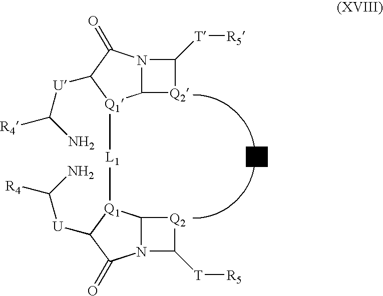

wherein:

R₁, R₄′, U, U′, Q₁, Q₁′, Q₂, Q₂′, T, T′, R₅, and R₅′ are defined as in claim 14; and

L₁ and L₂, independently, link position Q₁ with Q₁′ and Q₂ with Q₂′; or

a pharmaceutically acceptable salt thereof;
a compound of general formula (XIX):

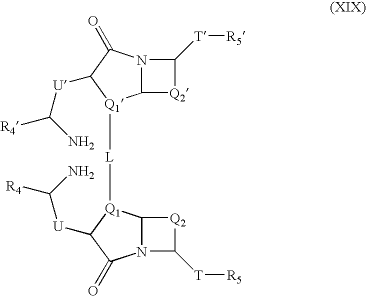

wherein:

R₄, R₄′, U, U′, Q₁, Q₁′, Q₂, Q₂′, T, T′, R₅, and R₅′ are defined as in claim 14; and

L covalently links position Q₁ with Q₁′; or

a pharmaceutically acceptable salt thereof;
a compound of general formula (XX):

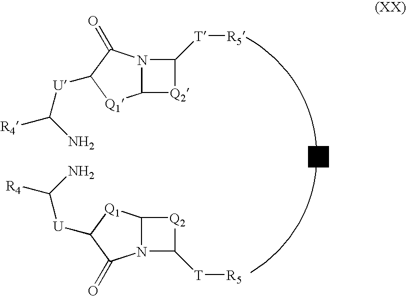

wherein:

R₄, R₅′, U, U′, Q₁, Q₁′, Q₂, Q₂′, T, T′, R₅, and R₅′ are defined as in claim 14; and

L covalently links position R₅ with R₅′; or

a pharmaceutically acceptable salt thereof; and
a compound of general formula (XXI):

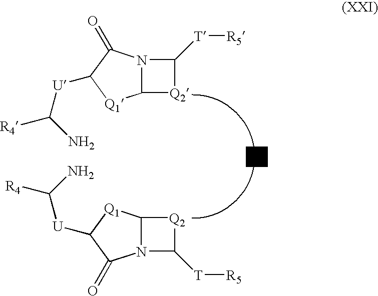

wherein:

R₄, R₄′, U, U′, Q₁, Q₁′, Q₂, Q₂′, T, T′, R₅ and R₅′ are defined as in claim 14; and

L covalently links position Q₂ with Q₂′; or

a pharmaceutically acceptable salt thereof

In certain embodiments, the compounds may further include a pharmaceutically acceptable excipient, and in certain other embodiments, the compounds of the invention may include a second therapeutic agent selected from chemotherapeutic agents, radiation, immunotherapeutic agents, photodynamic therapy agents, and combinations thereof.

### DESCRIPTION OF DRAWINGS

Not Applicable.

### DETAILED DESCRIPTION

It must be noted that, as used herein, and in the appended claims, the singular forms “a”, “an” and “the” include plural reference unless the context clearly dictates otherwise. Unless defined otherwise, all technical and scientific terms used herein, have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The terms “mimetic”, “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically produced peptides, recombinantly or chemically modified peptides, as well as non-peptide agents, such as small molecule drug mimetics, as further described below.

As used herein, the terms “pharmaceutically acceptable”, “physiologically tolerable” and grammatical variations thereof, as they refer to compositions, carriers, diluents and reagents, are used interchangeably and represent that the materials are capable of administration upon a mammal without the production of undesirable physiological effects such as nausea, dizziness, rash, or gastric upset.

“Providing” when used in conjunction with a therapeutic means to administer a therapeutic directly into or onto a target tissue, or to administer a therapeutic to a patient whereby the therapeutic positively impacts the tissue to which it is targeted.

As used herein, “subject”, “patient” or “individual” refers to an animal or mammal including, but not limited to, a human, dog, cat, horse, cow, pig, sheep, goat, chicken, monkey, rabbit, rat, or mouse, etc.

As used herein, the term “therapeutic” means an agent utilized to treat, combat, ameliorate, prevent or improve an unwanted condition or disease of a patient. Embodiments of the present invention are directed to promote apoptosis and, thus, cell death.

The terms “therapeutically effective amount” or “effective amount,” as used herein, may be used interchangeably and refer to an amount of a therapeutic compound component of the present invention. For example, a therapeutically effective amount of a therapeutic compound is a predetermined amount calculated to achieve the desired effect, i.e., to effectively promote apoptosis, preferably by eliminating IAP inhibition of apoptosis, more preferably by inhibiting an IAP binding to a caspase.

The terms “mimetics” or “peptidomimetics” are interchangeable and refer to synthetic compounds having a three-dimensional structure (i.e. a “core peptide motif”) based upon the three-dimensional structure of a selected peptide. The peptide motif provides the mimetic compound with the desired biological activity, i.e., binding to IAP, wherein the binding activity of the mimetic compound is not substantially reduced, and is often the same as or greater than the binding affinity of the native peptide on which the mimetic is modeled. For example, in the mimetics of the present invention, we have found that portions of compounds based on peptides can be non-peptide like. Peptidomimetic compounds can have additional characteristics that enhance their therapeutic application, such as increased cell permeability, greater affinity and/or avidity, and prolonged biological half-life.

“Optional” or “optionally” may be taken to mean that the subsequently described structure, event or circumstance may or may not occur, and that the description includes instances where the events occurs and instances where it does not.

“Alkyl” or “alkylene” unless otherwise specified, means a branched or unbranched, saturated aliphatic hydrocarbon group, having up to 12 carbon atoms. When used as part of another term, for example, “alkylamino,” the alkyl portion may be a saturated hydrocarbon chain. Examples of alkyl groups include methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl”, “C₁-C₄ alkyl”, and “alkyl of 1 to 4 carbon atoms” are synonymous and used interchangeably to mean methyl, ethyl, 1-propyl, isopropyl, cyclopropyl, 1-butyl, sec-butyl or t-butyl. Unless specified, substituted alkyl groups may contain one, two, three or four substituents which may be the same or different.

“Substituent” or “substituents” as used herein refer to a molecular group that replaces a hydrogen at any methyl group on a hydrocarbon. Substituents include, for example, halo, pseudohalo, hydroxy, protected hydroxy, trityloxy, carboxy, carbonyl, cyano, nitro, acyl, acyloxy, acetyl, acetoxy, carbamoyl, carbamoyloxy, allyl, allyloxy, oxo, thia, nitrile, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, cycloalkyloxy, heterocyclyl, heterocyclylalkyl, aryl, aryloxy, arylalkyl, aralkenyl, aralkynyl, heteroaryl, heteroaryloxy, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, heterocyclylsulfonyl, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N′,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, protected amino, aminoalkyl, aminothio, acylamino, aminosulfinyl, aminosulfonyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyl, alkylsulfonyloxy, alkylsulfonyl, alkylsulfonyloxy, arylsulfinyloxy, arylsulfonyloxy, hydroxysulfonyloxy, alkoxysulfonyloxy, aminosulfonyloxy, alkylaminosulfonyl alkylaminosulfonyloxy, alkylarylaminosulfonyloxy, alkylsulfonyl, arylsulfinyl, alkylsulfonylamino, arylsulfonyl, hydroxysulfonylo, alkoxysulfonyl, aminosulfonyl, alkylaminosulfonyl, dialkylaminosulfonyl, arylaminosulfonyl, diarylaminosulfonyl, and alkylarylaminosulfonyl, alkyloxycarbonylamino, allyloxycarbonyl, allyloxycarbonylamino, and the like. For example, particular substituted alkyls are substituted methyls, e.g., a methyl group substituted by the same substituents as the “substituted Cₙ-Cₘ alkyl” group. “Substituted alkyl” may include alkyloxymethyl, such as, methoxymethyl, ethoxymethyl, and t-butoxymethyl; halomethyl, such as, chloromethyl, bromomethyl, iodomethyl, and trifluoromethyl; hydroxymethyl; protected hydroxymethyl, such as, tetrahydropyranyloxymethyl; trityloxymethyl; cyanomethyl; nitromethyl; aminomethyl; carboxymethyl; alkyloxycarbonylmethyl; acetoxymethyl, carbamoyloxymethyl; all yloxycarbonylaminomethyl; propionyloxymethyl; acetoxymethyl; 6-hydroxyhexyl; 2,4-dichloro(n-butyl); 2 -amino(iso-propyl); 2-carbamoyloxyethyl; carbocycle group, such as, for example, cyclopropylmethyl, cyclobutylmethyl, cyclopentylmethyl, and cyclohexylmethyl groups, as well as the corresponding-ethyl, -propyl, -butyl, -pentyl, -hexyl groups, etc.

“Alkenyl” or “alkenylene” as used herein refers to an unsaturated, branched or unbranched, alphatic hydrocarbon having one or more double bond (—C═C—), and “alkynyl” or “alkynylene” as used herein refers to an unsaturated, branched or unbranched, alphatic hydrocarbon containing one or more triple bond (—C≡C—). Unsaturated hydrocarbons may have up to 12 carbon atoms and may be substituted by one or more of any of the substituents described hereinabove. When used as part of another term, for example, “alkenylamino” and “alkynylamino” the alkyl portion may be an unsaturated hydrocarbon chain.

“Amino” denotes primary (i.e. —NH₂), secondary (i.e. —NRH), and tertiary (i.e. —NRR) amines. Particular secondary and tertiary amines include, but are not limited to, alkylamine, dialkylamine, arylamine, diarylamine, arylalkylamine and diarylalkylamine including, for example, methylamine, ethylamine, propylamine, isopropylamine, phenylamine, benzylamine, dimethylamine, diethylamine, dipropylamine and disopropylamine.

“Aryl”, when used alone or as part of another term, means a fused or unfused carbocyclic aromatic group having a designated number of carbon atoms, or if no number is designated, up to 14 carbon atoms. Particular aryl groups include phenyl, naphthyl, biphenyl, phenanthrenyl, naphthacenyl, and the like (see, Lang's Handbook of Chemistry 13^{th} ed. (Dean, J. A., ed.) Table 7-2 [1985]). Substituted phenyl or substituted aryl denotes a phenyl or aryl group substituted with one, two, three, four or five substituents chosen from those described above, for example, halogen (F, Cl, Br, I), hydroxy, protected hydroxy. cyano, nitro, alkyl (such as C₁-C₆ alkyl), alkoxy (such as, C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, arylsulfonylamino, heterocyclylsulfonylamino, heterocyclyl, or aryl, and one or more methyne (CH) and/or methylene (CH₂) groups in these substituents may be substituted with a group similar to those described above. Examples of “substituted phenyls” that may be utilized in embodiments of the invention include, but are not limited to, mono- or di-halo-phenyl, such as, 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl, and the like; mono- or di-hydroxyphenyl, such as, 4-hydroxyphenyl, 3-hydroxyphenyl, 2,4-dihydroxyphenyl, protected-hydroxy derivatives thereof, and the like; nitrophenyl, such as, 3- or 4-nitrophenyl; cyanophenyl, for example, 4-cyanophenyl; mono- or di-lower alkyl-phenyl group, such as, 4-methylphenyl, 2,4-dimethylphenyl, 2-methylphenyl, 4-(iso-propyl)phenyl, 4-ethylphenyl, 3-(n-propyl)phenyl, and the like; a mono- or di-alkoxy-phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; mono- or di-carboxyphenyl or protected carboxy phenyl, such as, 4-carboxyphenyl; mono- or di-hydroxymethyl-phenyl or protected hydroxymethyl phenyl, such as 3-(protected hydroxymethyl)phenyl or 3,4-di(hydroxymethyl)phenyl; mono- or di-(aminomethyl) phenyl or protected aminomethyl phenyl, such as 2-(aminomethyl)phenyl or 2,4-(protected aminomethyl)phenyl; or mono- or di-(N-(methylsulfonylamino))phenyl, such as, 3-(N-methylsulfonylamino)phenyl. In addition, “substituted phenyl” may represent di-substituted phenyl groups where the substituents are different, such as, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as tri-substituted phenyl groups where the substituents are different, such as, for example, 3-methoxy-4-benzyloxy-6-methyl sulfonylamino, 3-methoxy-4-benzyloxy-6-phenyl sulfonylamino and the like and tetra-substituted phenyl groups where the substituents are different, such as, for example, 3-methoxy-4-benzyloxy-5-methyl-6-phenyl sulfonylamino. Particular substituted phenyl groups include 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl), and benzyloxy-6-methyl sulfonyl aminophenyl groups. Fused aryl rings may also be substituted with one or more of any of the substituents specified herein, for example, fused aryl groups may contain 1, 2 or 3 substituents in the same manner as substituted alkyl groups.

“Heterocyclic group”, “heterocyclic”, “heterocycle”, “heterocyclyl”, or “heterocyclo” alone, and when used as a moiety in a complex group such as a heterocycloalkyl group, are used interchangeably and refer to any mono-, bi-, or tri-cyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen). In a particular embodiment, the group incorporates 1 to 4 heteroatoms. Typically, a 5-member ring has 0 to 2 double bonds and a 6- or 7-member ring has 0 to 3 double bonds; and the nitrogen or sulfur heteroatoms may optionally be oxidized (e.g. SO, SO₂), and any nitrogen heteroatom may optionally be quaternized. Particular non-aromatic heterocycles include morpholinyl(morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl, and piperidinyl. A “heterocycloalkyl” group is a heterocycle group as defined above, covalently bonded to an alkyl group as defined above. Particular 5-membered heterocycles containing a sulfur or oxygen atom and one to three nitrogen atoms include thiazolyl, such as thiazol-2-yl and thiazol-2-yl N-oxide, thiadiazolyl, such as 1,3,4-thiadiazol-5-yl and 1,2,4-thiadiazol-5-yl, oxazolyl, such as, oxazol-2-yl, and oxadiazolyl, such as 1,3,4-oxadiazol-5-yl, and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl, 1,2,3-triazol-5-yl, and 1,2,4-triazol-5-yl, and tetrazolyl such as 1H-tetrazol-5-yl. Particular benzo-fused 5-membered heterocycles are benzoxazol-2-yl, benzthiazol-2-yl, and benzimidazol-2-yl. Particular 6-membered heterocycles contain one to three nitrogen atoms and, optionally, a sulfur or oxygen atom, for example pyridyl, such as, pyrid-2-yl, pyrid-3-yl, and pyrid-4-yl; pyrimidyl, such as, pyrimid-2-yl and pyrimid-4-yl; triazinyl, such as, 1,3,4-triazin-2-yl and 1,3,5-triazin-4-yl; pyridazinyl, such as, pyridazin-3-yl, and pyrazinyl. Substituents for optionally substituted heterocycles, and further examples of the 5- and 6-membered ring systems discussed above, can be found in U.S. Pat. No. 4,278,793 to W. Druckheimer et al.

“Heteroaryl” alone and when used as a moiety in a complex group such as a heteroarylalkyl group, refers to any mono-, bi-, or tri-cyclic aromatic ring system having the number of atoms designated where at least one ring is a 5-, 6- or 7-membered ring containing from one to four heteroatoms selected from the group nitrogen, oxygen, and sulfur (see Lang's Handbook of Chemistry, supra). Included in the definition are any bicyclic groups where any of the above heteroaryl rings are fused to a benzene ring. The following ring systems are examples of the heteroaryl (whether substituted or unsubstituted) group denoted by the term “heteroaryl”: thienyl, furyl, imidazolyl, pyrazolyl, triazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example, benzoxazolyl, benzofuryl, benzothiazolyl, benzothiadiazolyl, benzotriazolyl, benzoimidazolyl, and indolyl. Particular “heteroaryls” include: 1,3-thiazol-2-yl, 4-(carboxymethyl)-5-methyl, 1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes: 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H- tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl.

A “linker” is a bond or linking group whereby two chemical moieties, such as, monomers of an active compound, are directly covalently linked to one another or are indirectly linked via a third chemical moiety to form a homo- or heterodimer. The compounds set forth herein may include a single linker linking the two chemical moieties, or more than one linker linking the two chemical moieties at one or more position independently on each of the two chemical moieties. A “linker” (L, L₁ or L₂) may be a single or double covalent bond or a branched or unbranched, substituted or unsubstituted, hydrocarbon chain of 1 to about 100 atoms, typically, 1 to about 20 atoms, having a molecular weight up to about 500 MW. For example, a linker can be a bond, alkylene, alkenylene, alkynylene, cycloalkylene, cycloalkylalkylene, heterocycloalkylene, heterocycloalkylalkylene, aryl, arylalkylene, arylalkylalkylene, heteroaryl, or heteroarylalkylene, or an optionally-substituted alkylene, alkenylene, alkynylene cycloalkylene, cycloalkylalkylene, heterocycloalkylene, heterocycloalkylalkylene, aryl, arylalkylene, arylalkylalkylene, heteroaryl, or heteroarylalkylene, of 2 to 12 atoms where one or more carbon atoms can be replaced with N, O, or S or an amino, substituted amino, oxygen atom, sulfide (—S—), sulfoxide (—SO—), sulfone (—SO₂—), or disulfide (—SS—) group. Illustrative linkers and linking groups are described in U.S. Patent Publication No. 20050197403, as well as in U.S. patent application Ser. No. 11/363,387, filed Feb. 27, 2006, both of which are incorporated herein by reference as though fully set forth. For example, particular “linkers” include, but are not limited to, —CH₂CH₂—, —CH₂CH₂CH₂—, —CH═CH—, 1,4-phenyl, 2,5-thiophenyl, —CH(OH)CH(OH)—, —CH₂CH—O—CHCH₂—, and —CH₂C═CC′CCH₂—.

The term “homodimer” as used herein refers to a compound composed of two covalently bound monomeric units of a chemical moiety wherein the monomeric units are identical.

The term “heterodimer” as used herein refers to a compound composed of two covalently bound monomeric units of a chemical moiety wherein the monomeric units are different. For example, one monomeric unit of a heterodimer may include a substituent that is different from the other monomeric unit at one or more position.

“Inhibitor” means a compound which reduces or prevents a particular interaction or reaction. For example, the binding of IAP proteins to caspase proteins reduces or prevents the inhibition of apoptosis by an IAP protein.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, arylaliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

The present invention is generally directed to Smac peptidomimetics (herein referred to as “Smac mimetics” or “a Smac mimetic”) and the uses of Smac mimetics. One embodiment of the invention is a therapeutic composition including a Smac mimetic. In another embodiment, Smac mimetics act as chemopotentiating or chemotherapeutic agents. The term “chemopotentiating agent” as used herein refers to an agent that acts to increase the sensitivity of an organism, tissue, or cell to a chemical compound, or treatment namely “chemotherapeutic agents” or “chemo drugs” or radiation treatment. Therefore, a further embodiment of the invention is the therapeutic composition of a Smac mimetic, which acts as a chemopotentiating agent, and a biological agent, chemotherapeutic agent or radiation. Another embodiment of the invention is a method of inhibiting tumor growth in vivo by administering a Smac mimetic. Yet another embodiment is a method of inhibiting tumor growth in vivo by administering a Smac mimetic and a biologic agent, chemotherapeutic agent or radiation. Still another embodiment of the invention is a method of treating an individual, such as, for example, patient with cancer, by administering Smac mimetics of the present invention alone, or in combination with, a biological agent, chemotherapeutic agent or radiation.

In various embodiments of the invention, in situ cells or pathogenic cells, in an individual, may be treated with a Smac mimetic or a Smac mimietic in combination with a secondary agent, such as, a biological agent, chemotherapeutic agent or radiation. In such embodiments, the contacting step is affected by administering a pharmaceutical composition including a therapeutically effective amount of the Smac mimetic, wherein the individual may be subject to concurrent or antecedent radiation or chemotherapy for treatment of a neoproliferative pathology. Pathogenic cells may be of a tumor such as, but not limited to, bladder cancer, breast cancer, prostate cancer, lung cancer, pancreatic cancer, gastric cancer, colon cancer, ovarian cancer, renal cancer, hepatoma, melanoma, lymphoma, sarcoma, and combinations thereof.

In addition to apoptosis defects found in tumors, defects in the ability to eliminate self-reactive cells of the immune system due to apoptosis resistance may be considered to play a key role in the pathogenesis of autoimmune diseases. Autoimmune diseases are characterized in that the cells of the immune system produce antibodies against its own organs and molecules or directly attack tissues resulting in the destruction of these tissues. Failure of these self-reactive cells to undergo apoptosis leads to the manifestation of the disease. Defects in apoptosis regulation have been identified in autoimmune diseases such as systemic lupus erthematosus, or rheumatoid arthritis.

In some embodiments of the invention, pathogenic cells may be those cells effected by an autoimmune disease or any disease whose symptoms include production of cells that are resistant to apoptosis. In at least one embodiment, affected cells are resistant to apoptosis due to the expression or overexpression of members of the Bcl-2 family of caspases. Examples of such autoimmune diseases include, but are not limited to, collagen diseases, such as, rheumatoid arthritis, systemic lupus erythematosus, Sharp's syndrome, CREST syndrome, calcinosis, Raynaud's syndrome, esophageal dysmotility, telangiectasia, dermatomyositis, vasculitis (Morbus Wegener's), and Sjögren's syndrome; renal diseases, such as, Goodpasture's syndrome, rapidly-progressing glomerulonephritis, and membrano-proliferative glomerulonephritis type II; endocrine diseases, such as, type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyreosis, Hashimoto's thyroiditis, and primary myxedema; skin diseases, such as, pemphigus vulgaris, bullous pemphigoid, herpes gestationis, epidermolysis bullosa, and erythema multiforme major; liver diseases, such as, primary biliary cirrhosis, autoimmune cholangitis, autoimmune hepatitis type-1, autoimmune hepatitis type-2, primary sclerosing cholangitis; neuronal diseases, such as, multiple sclerosis, myasthenia gravis, myasthenic Lambert-Eaton syndrome, acquired neuromyotony, Guillain-Barré syndrome (Müller-Fischer syndrome), stiff-man syndrome, cerebellar degeneration, ataxia, opsoklonus, sensoric neuropathy, and achalasia; blood diseases, such as, autoimmune hemolytic anemia and idiopathic thrombocytopenic purpura (Morbus Werlhof); and infectious diseases with associated autoimmune reactions, such as, AIDS, Malaria, and Chagas disease.

It has been demonstrated in accordance with the present invention that the IAP-binding peptides or mimetics, thereof, are capable of potentiating apoptosis of cells. The mimetics described herein are suitably small, and since structural features in relation to the IAP binding groove are well-characterized, a wide variety of mimetic compounds may be synthesized. Mimetics of the core IAP-binding portions are preferred. Added advantages of compounds of this size include improved solubility in aqueous solution and ease of delivery to selected targets in vivo.

The following compounds are illustrative of IAP-binding compounds that may be prepared as dimers and dimers of these IAP-binding compounds. Thus, various embodiments of the invention include these dimers. Such dimers can be prepared using any synthetic technique available to persons of ordinary skill in the art, such as, for example, the dimeric Smac peptidomimetics disclosed in U.S. patent application Ser. No. 11/363,387, filed Feb. 27, 2006, which provides guidance on preparation of the dimers of the instant invention.

Various embodiments of the invention include homodimers and heterodimer of compounds having monomeric units of general formula (I):

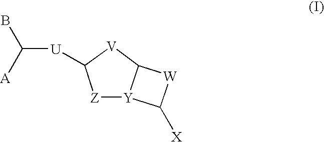

wherein:

each A is, independently, NR₁R₂, or N⁺R₁R₂R₃;

each R₁, R₂, and R₃ is, independently, hydrogen or optionally substituted alkyl, alkenyl, or alkynyl, wherein one or more carbons are optionally replaced by C═O, C═S, or a heteroatom selected from O, S, and N and wherein one or more hydrogens are optionally replaced by fluorine, a branched or unbranched alkyl or cycloalkyl, an optionally substituted aryl, arylalkyl, heteroaryl, heteroarylalkyl, OR₄, SR₄, or NR₄R₅;

each R₄ and R₅ are, independently, hydrogen or optionally substituted alkyl, alkenyl, or alkynyl, wherein one or more carbons are optionally replaced by a heteroatom selected from O, S, and N, or optionally substituted aryl, arylalkyl, heteroaryl, or heteroarylalkyl; or any two of R₁, R₂, and R₃ taken together with the nitrogen to which they are attached to form a heterocyclic group, in which one or more carbon atoms are optionally replaced by C═O, C═S, or a heteroatom selected from O, S, and N, with the proviso that the heteroatom is separated from the nitrogen atom by at least two carbons;

each B is, independently, optionally substituted alkyl, alkenyl, or alkynyl, wherein one or more hydrogens are optionally replaced by fluorine;

each U is, independently, CONH, C(O)O, C(S)O, C(S)NH, C(NH)NH, (CH₂)₁₋₅, or the inverse, thereof, wherein one or more carbons are optionally replaced by a heteroatom selected from O, S, and N;

each V and W is, independently, (CH₂)₁₋₅, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR_{I}, or NR₄R₅;

each X is, independently, optionally substituted alkyl, alkenyl, alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;

each Y is, independently, CH or N;

each Z is, independently, CH₂, C═O, C═S, CHSR, CHOR, or CHNR; and

each R is, independently, hydrogen or optionally substituted alkyl, alkenyl, or alkynyl; or

pharmaceutically acceptable salts thereof.

Other embodiments of the invention include compounds having structure of general formula (II):

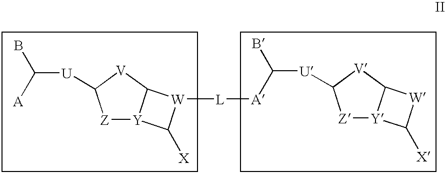

wherein:

A and A′ are each, independently, NR₁R₂ or N⁺R₁R₂R₃;

R₁, R₂, and R₃ are each, independently, hydrogen or optionally substituted alkyl, alkenyl, or alkynyl, wherein one or more carbons are optionally replaced by C═O, C═S, or a heteroatom selected from O, S, and N, and one or more hydrogens are optionally replaced by fluorine, a branched or unbranched alkyl or cycloalkyl, an optionally substituted aryl, arylalkyl, heteroaryl, heteroarylalkyl, OR₄, SR₄, or NR₄R₅;

R₄ and R₅ are each, independently, hydrogen or optionally substituted alkyl, alkenyl, or alkynyl, in which one or more carbons are optionally replaced by a heteroatom selected from O, S, and N, or optionally substituted aryl, arylalkyl, heteroaryl, or heteroarylalkyl; or any two of R₁, R₂, and R₃ taken together with the nitrogen to which they are attached to form a heterocyclic group, wherein one or more carbon atoms are optionally replaced by C═O, C═S, or a heteroatom selected from O, S, and N, with the proviso that the heteroatom is separated from the nitrogen atom by at least two carbons;

B and B′ are each, independently, optionally substituted alkyl, alkenyl, or alkynyl, wherein one or more hydrogens are optionally replaced by fluorine;

U and U′ are each, independently, CONH, C(O)O, C(S)O, C(S)NH, C(NH)NH, (CH₂)₁₋₅, or the inverse wherein one or more carbons are optionally replaced by a heteroatom selected from O, S, and N;

V, V′, W and W′ are each, independently, (CH₂)₁₋₅, wherein one or more carbons are optionally replaced by C═O, C═S, or a heteroatom selected from O, S, and N, and one or more hydrogens in CH₂ groups are optionally replaced by a branched or unbranched alkyl or cycloalkyl, an optionally substituted aryl, arylalkyl, heteroaryl, heteroarylalkyl, OR₄, SR₄, or NR₄R₅;

X and X′ are each, independently, optionally substituted C₁₋₁₈-alkyl, C₂₋₁₈-alkenyl, or C₂₋₁₈-alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;

Y and Y′ are each, independently, CH or N;

Z and Z′ are each, independently, CH₂, C═O, C═S, CHSR, CHOR, or CHNR;

each R is, independently, hydrogen, or optionally substituted alkyl, alkenyl, or alkynyl; and

L is one or more bonds or one or more linkers covalently linking one or more of positions W, and X, with V′, W′, and X′; and

pharmaceutically acceptable salts thereof.

In still other embodiments the compounds of the invention may have a formula selected from formula (III):

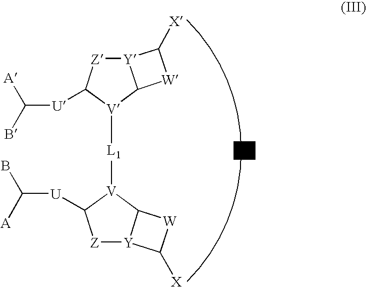

wherein:

A, A′, B, B′, U, U′, Z, Z′, Y, Y′, V, V′, W, W′, X, and X′ are defined as described above; and

L₁ and L₂ are bonds or linkers that, independently, link position V with V′ and X with X′; and

pharmaceutically acceptable salts thereof;
formula (IV):

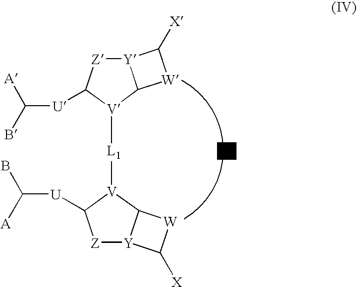

wherein:

A, A′, B, B′, U, U′, Z, Z′, Y, Y′, V, V′, W, W′, X, and X′ are defined as described above; and

L₁ and L₂ are bonds or linkers that, independently, link position V with V′ and W with W′; and

pharmaceutically acceptable salts thereof;
general formula (V):

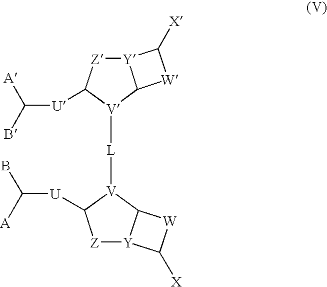

wherein:

A, A′, B, B′, U, U′, Z, Z′, Y, Y′, V, V′, W, W′, X, and X′ are defined as described above; and

L is a bond or linker that covalently links position V with V′; and

pharmaceutically acceptable salts thereof;
formula (VI):

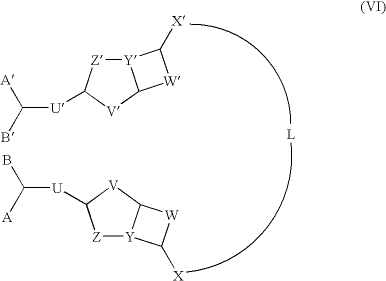

wherein:

A, A′, B, B′, U, U′, Z, Z′, Y, Y′, V, V′, W, W′, X, and X′ are defined as described above; and

L is a bond or linker that links position X with X′; and

pharmaceutically acceptable salts thereof; and

formula (VII):

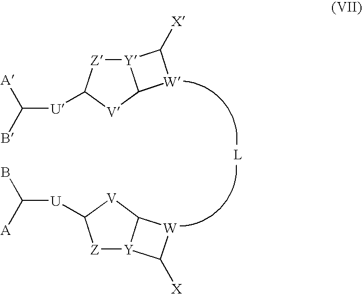

wherein:

A, A′, B, B′, U, Z, Z′, Y, Y′, V, V′, W, W′, X, and X′ are defined as described above; and

L is a bond or linker that links position W with W′; and

pharmaceutically acceptable salts thereof.

Various other embodiments of the invention include compounds of a homodimer or heterodimer having monomeric units of formula (VIII):

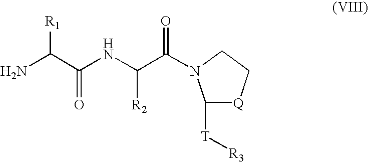

wherein:

each R₁ is, independently, alkyl or haloalkyl;

each R₂ is, independently, branched or unbranched alkyl or cycloalkyl, or substituted or unsubstituted aryl, arylalkyl, heteroaryl, or heteroarylalkyl;

each R₃ is, independently, branched or unbranched alkyl or cycloalkyl, or substituted or unsubstituted aryl, arylalkyl, heteroaryl, or heteroarylalkyl;

each S is, independently, (CH₂)₀₋₃ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

each T is, independently, CONH, CH₂O, NHCO, (CH₂)₁₋₄, (CH₂)₁₋₃CONH(CH₂)₀₋₃, (CH₂)₁₋₃, (CH₂)₀₋₃, (CH₂)₁₋₃NH(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is a branched or unbranched alkyl, cycloalkyl or substituted or unsubstituted aryl, arylalkyl, heteroaryl, or heteroarylalkyl teroaryl; and

pharmaceutically acceptable salts thereof.

The compound of claim 7, having the formula (IX):

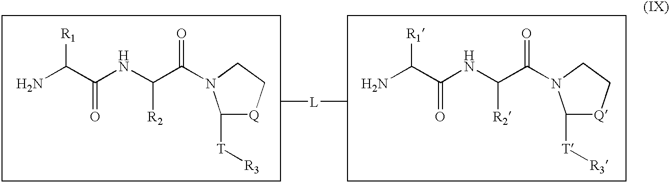

wherein:

R₁ and R₁′ are each, independently, alkyl or haloalkyl;

R₂ and R₂′ are each, independently, branched or unbranched alkyl or cycloalkyl, or substituted or unsubstituted aryl, arylalkyl, heteroaryl, or heteroarylalkyl;

R₃ and R₃′ are each, independently, branched or unbranched alkyl or cycloalkyl, or substituted or unsubstituted aryl, arylalkyl, heteroaryl, or heteroarylalkyl;

Q and Q′ are each, independently, (CH₂)₀₋₃ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

T and T′ are each, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and

L is one or more linkers covalently linking one or more of the positions R₂, R₃, Y, with R₂′, R₃′, Y′; and

pharmaceutically acceptable salts thereof.

In some embodiments, the compounds of the invention may have a formula selected from compounds of formula (X):

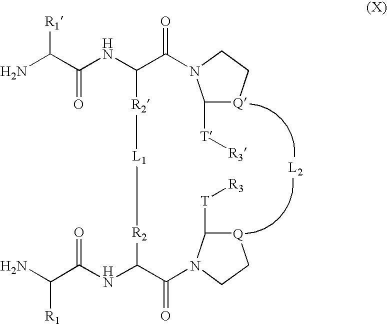

wherein:

R₁, R₁′, R₂, R₂′, R₃, R₃′, Q, Q′, T, and T′ are defined as described above; and

L₁ and L₂ are bonds or linkers that, independently, link position R₂ with R₂′ and Q with Q′; and

pharmaceutically acceptable salts thereof;
formula (XI):

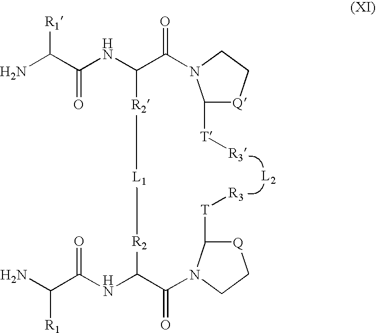

wherein:

R₁, R₁′, R₂, R₂′, R₃, R₃′, Q, Q′, T, and T′ are defined as described above; and

L₁ and L₂ are bonds or linkers that, independently, link position R₂ with R₂′ and R₃ with R₃′; and

pharmaceutically acceptable salts thereof;
formula (XII):

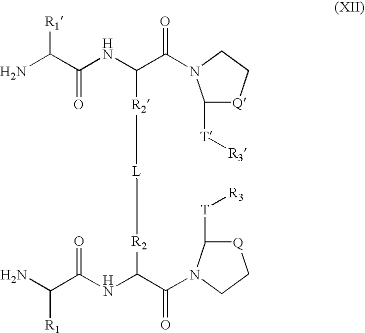

wherein:

R₁, R₁′, R₂, R₂′, R₃, R₃′, Q, Q′, T, and T′ are defined as described above; and

L is a bond or linker that links position R₂ with R₂′; and

pharmaceutically acceptable salts thereof;
formula (XIII):

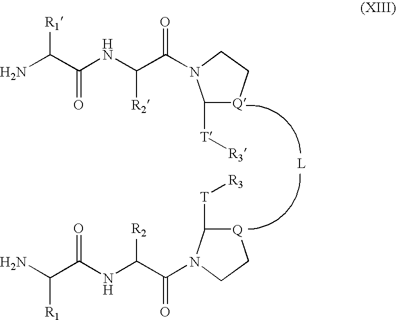

wherein:

R₁, R₁′, R₂, R₂′, R₃, R₃′, Q, Q′, Z, and Z′ are defined as described above; and

L is a bond or linker that links position Q with Q′; or

pharmaceutically acceptable salts thereof; and
formula (XIV):

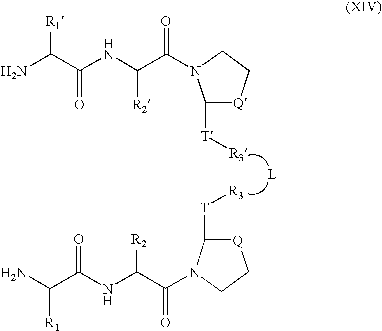

wherein:

R₁, R₁′, R₂, R₂′, R₃, R₃′, Q, T, and T′ are defined as described above; and

L is a bond or linker that links position R₃ with R₃′; and

pharmaceutically acceptable salts thereof.

In other embodiments, the compounds of the invention include homodimers and heterodimers having monomeric units of general formula (XV):

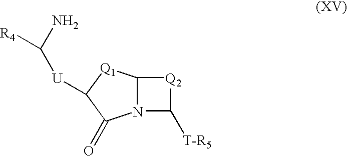

wherein:

each R₄ is, independently, alkyl or haloalkyl;

each R₅ is, independently, branched or unbranched alkyl or cycloalkyl, or substituted or unsubstituted aryl, arylalkyl, heteroaryl, or heteroarylalkyl;

each U is, independently, CONH, CH₂O, CH₂NH, CH₂S, or (CH₂)₁₋₃;

each Q₁ is, independently, (CH₂)₁₋₅ wherein one or more carbons can be replaced by one or more heteroatoms selected from O, S, and N; and one or more hydrogens in CH₂ groups can be replaced by a branched or unbranched alkyl or cycloalkyl, or substituted or unsubstituted aryl, arylalkyl, heteroaryl, or heteroarylalkyl;

each Q₂ is, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

each T is, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and

pharmaceutically acceptable salts thereof.

In still other embodiments, the compounds of the invention may be of general formula (XVI):

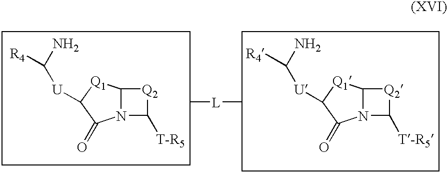

wherein:

R₄ and R₄′ are each, independently, alkyl or haloalkyl;

R₅ and R₅′ are each, independently, branched or unbranched alkyl or cycloalkyl, or substituted or unsubstituted aryl, arylalkyl, heteroaryl, or heteroarylalkyl;

U and U′ are each, independently, CONH, CH₂O, CH₂NH, CH₂S, or (CH₂)₁₋₃;

Q₁ and Q₁′ are each, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

Q₂ and Q₂′ are each, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and

T and T′ are each, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and

L is one or more linkers covalently linking one or more of the positions Y₁, Y₂, R₂, with Y₁′, Y₂′, R₂′; and

pharmaceutically acceptable salts thereof.
18. In certain embodiments, the compounds of the invention may be selected from compounds of general formula (XVII):

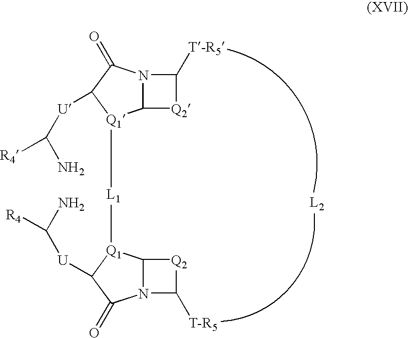

wherein:

R₄, R₄′, U, U′, Q₁, Q₁′, Q₂, Q₂′, T, T′, R₅, and R₅′ are defined as described above; and

L₁ and L₂ are bonds or linkers that, independently, link position Q₁ with Q₁′ and R₅ with R₅′; and

pharmaceutically acceptable salts thereof;

formula (XVIII):

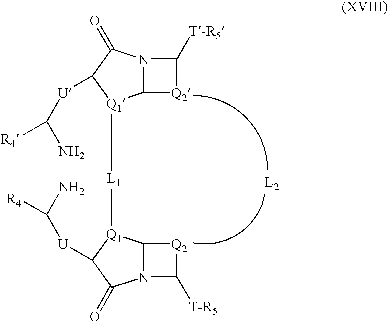

wherein:

R₄, R₄′, U, U′, Q₁, Q₁′, Q₂, Q₂′, T, T′, R₅, and R₅′ are defined as described above; and

L₁ and L₂ are bonds or linkers that, independently, link position Q₁ with Q₁′ and Q₂ with Q₂′; and

pharmaceutically acceptable salts thereof;

formula (XIX):

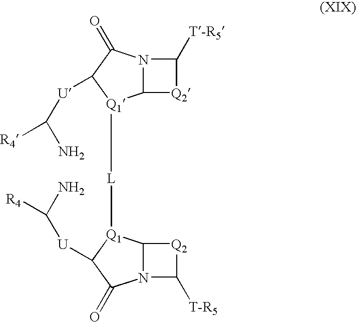

wherein:

R₄, R₄′, U, U′, Q₁, Q₂, Q₂′, T, T′, R₅, and R₅′ are defined as described above; and

L is a bond or linker that links position Q₁ with Q₁′; and

pharmaceutically acceptable salts thereof;

formula (XX):

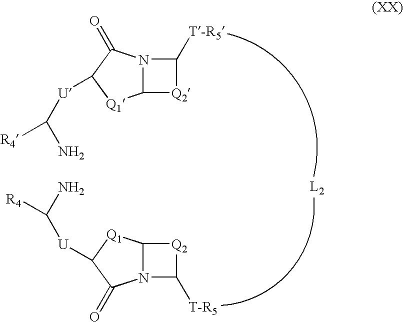

wherein:

R₄, R₅′, U, U′, Q₁, Q₁′, Q₂, Q₂′, T, T′, R₅, and R₅′ are defined as described above; and

L is a bond or linker that links position R₅ with R₅′; and

pharmaceutically acceptable salts thereof; and

formula (XXI):

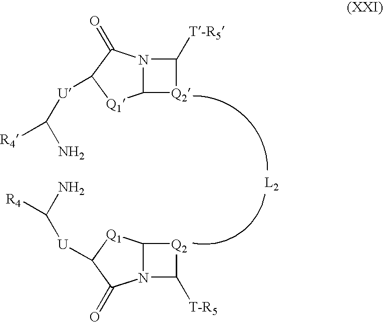

wherein:

R₄, R₄′, U, Q₁, Q₁′, Q₂, T, T′, R₅ and R₅′ are defined as described above; and

L is a bond or linker that links position Q₂ with Q₂′; or

pharmaceutically acceptable salts thereof

Mimetic, specifically, peptidomimetic design strategies are readily available in the art and can be easily adapted for use in the present invention (see, e.g., Ripka & Rich, Curr. Op. Chem. Biol. 2, 441-452, 1998; Hruby et al., Curr. Op. Chem. Biol. 1, 114-119, 1997; Hruby & Bake. Curr. Med. Chem. 9, 945-970, 2000). One class of mimetic mimics a backbone that is partially or completely non-peptide, but mimics the peptide backbone atom-for-atom and comprises side groups that likewise mimic the functionality of the side groups of the native amino acid residues. Several types of chemical bonds, e.g. ester, thioester, thioamide, retroamide, reduced carbonyl, dimethylene, and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a non-peptide scaffold to serve as “topographical” mimetics of the original peptide (Ripka & Rich, 1998, supra).

In one embodiment, the Smac mimetics of the invention are modified to produce peptide mimetics by replacement of one or more naturally occurring side chains of the 20 genetically encoded amino acids, or D-amino acids, with other side chains, for instance with groups, such as, alkyl, lower alkyl, cyclic 4-, 5-, 6-, to 7-membered alkyl, amide, amide lower alkyl, amide di-(lower alkyl), lower alkoxy, hydroxy, carboxy, and the lower ester derivatives thereof, and with 4-, 5-, 6-, to 7-membered heterocycles. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include furazanyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

Pharmaceutical compositions The subject compositions encompass pharmaceutical compositions including a therapeutically effective amount of a Smac mimetic in dosage form and a pharmaceutically acceptable carrier, wherein the Smac mimetic inhibits the activity of an Inhibitor of Apoptosis protein (IAP), thus promoting apoptosis. In another embodiment, the compositions include a therapeutically effective amount of a Smac mimetic in dosage form and a pharmaceutically acceptable carrier in combination with a chemotherapeutic and/or radiotherapy, wherein the Smac mimetic inhibits the activity of an IAP, thus promoting apoptosis and enhancing the effectiveness of the chemotherapeutic and/or radiotherapy.

In an embodiment of the invention, a therapeutic composition for promoting apoptosis can be a therapeutically effective amount of a Smac mimetic which binds to at least one IAP. In one embodiment, the IAP can be XIAP. In another embodiment, the IAP can be ML-IAP, and in yet another embodiment, the IAP can be cIAP-1 or cIAP-2. In further embodiments, the IAP can be multiple IAPs.

Embodiments of the invention also include methods for treating a patient having a condition characterized by inhibited apoptosis, wherein administration of a therapeutically effective amount of a Smac mimetic is delivered to the patient, and the Smac mimetic binds to at least one IAP. In one embodiment, the IAP can be XIAP. In another embodiment the IAP can be ML-IAP, and in still another embodiment, the IAP can be cIAP-1 or cIAP-2. In further embodiments, the IAP can be multiple IAPs.

In one embodiment of the invention, an additional chemotherapeutic agent (infra) or radiation may be administered prior to, along with, or following administration of the Smac mimetic. Exemplary chemotherapeutic agent, include, but are not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

In various embodiments, the Smac mimetics of the invention may be combined with a pharmaceutically acceptable carrier or excipient, and in some embodiments, the Smac mimetics of the invention may be combined with an additional chemotherapeutic agent and a pharmaceutically acceptable carrier or excipient. The term “pharmaceutically-acceptable carrier” as used herein means one or more compatible solid or liquid fillers, diluents or encapsulating substances which are suitable for administration into a human. The term “carrier” or “excipient” denotes an organic or inorganic ingredient, natural or synthetic, with which the active ingredient is combined to facilitate the application. The components of the pharmaceutical compositions are also capable of being co-mingled with the molecules of the present invention, and with each other, in a mariner such that there is no interaction which would substantially impair the desired pharmaceutical efficacy.

The delivery systems of the invention are designed to include time-released, delayed release or sustained release delivery systems such that the delivering of the Smac mimetic occurs prior to, and with sufficient time, to cause sensitization of the site to be treated. A Smac mimetic may be used in conjunction with radiation and/or additional anti-cancer chemical agents (infra). Such systems can avoid repeated administrations of the Smac mimetic compound, increasing convenience to the subject and the physician, and may be particularly suitable for certain compositions of the present invention.

Many types of release delivery systems are available and known to those of ordinary skill in the art. They include, but are not limited to, polymer base systems, such as, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems including: lipids including sterols, such as cholesterol, cholesterol esters and fatty acids or neutral fats, such as mono-, di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034, and 5,239,660 and (b) diffusional systems in which an active component permeates at a controlled rate from a polymer, such as described in U.S. Pat. Nos. 3,832,253, and 3,854,480. In addition, pump-based hardware delivery systems can be used, some of which are adapted for implantation.

Use of a long-term sustained release implant may be desirable. Long-term release is used herein, and means that the implant is constructed and arranged to deliver therapeutic levels of the active ingredient for at least about 30 days, and preferably about 60 days. Long-term sustained release implants are well-known to those of ordinary skill in the art and include some of the release systems described above.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions may be prepared by uniformly and intimately bringing the active compound into association with a liquid carrier, a finely divided solid carrier, or both, and then, if necessary, shaping the product.

Compositions suitable for parenteral administration conveniently include a sterile aqueous preparation of a Smac mimetic which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids, such as oleic acid, may be used in the preparation of injectables. Carrier formulation suitable for oral, subcutaneous, intravenous, intramuscular, etc. administrations can be found, for example, in Remington's Pharmaceutical Sciences, Mack Publishing Co., Easton, Pa. which is incorporated herein in its entirety by reference thereto.

Administration of Smac peptidomimetics The Smac peptidomimetics of the invention may be administered in effective amounts. An effective amount is that amount of a preparation that alone, or together with further doses, produces the desired response. This may involve only slowing the progression of the disease temporarily, although it may involve halting the progression of the disease permanently or delaying the onset of or preventing the disease or condition from occurring. This can be monitored by routine methods known and practiced in the art. Generally, doses of active compounds may be from about 0.01 mg/kg per day to about 1000 mg/kg per day, and in some embodiments, the dosage may be from 50-500 mg/kg. In various embodiments, the compounds of the invention may be administered intravenously, intramuscularly, or intradermally, and in one or several administrations per day. The administration of the Smac peptidomimetic can occur simultaneous with, subsequent to, or prior to chemotherapy or radiation.

In general, clinical trials will determine specific ranges for optimal therapeutic effect for each therapeutic agent and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the potency of the Smac mimetic administered, the duration of the treatment and the severity of the disease being treated. For example, a dosage regimen of the Smac mimetic to reduce tumor growth can be oral administration of from about 1 mg to about 2000 mg/day, preferably about 1 to about 1000 mg/day, more preferably about 50 to about 600 mg/day, in two to four divided doses. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient's tolerance permits. Multiple doses per day are contemplated to achieve appropriate systemic levels of compounds. Generally, a maximum dose is used, that is, the highest safe dose according to sound medical judgment. Those of ordinary skill in the art will understand, however, that a patient may insist upon a lower dose or tolerable dose for medical reasons, psychological reasons or for virtually any other reason.

Embodiments of the invention also include a method of treating a patient with cancer or an autoimmune disease by promoting apoptosis wherein administration of a therapeutically effective amount of a Smac mimetic and the Smac mimetic binds to at least one IAP. In one embodiment, the IAP can be XIAP. In another embodiment, the IAP can be ML-IAP, and in still another embodiment, the IAP can be cIAP-1 or cIAP-2. In further embodiments, the IAP can be multiple IAPs. The method may further include concurrent administration of a chemotherapeutic agent including, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

A variety of administration routes are available. The particular mode selected will depend, of course, upon the particular chemotherapeutic drug selected, the severity of the condition being treated, and the dosage required for therapeutic efficacy. The methods of the invention may be practiced using any mode of administration that is medically acceptable, meaning any mode that produces effective levels of active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes are particularly suitable for purposes of the present invention.

In one aspect of the invention, a Smac mimetic as described herein, with or without additional biological or chemotherapeutic agents or radiotherapy, does not adversely affect normal tissues, while sensitizing tumor cells to the additional chemotherapeutic/radiation protocols. While not wishing to be bound by theory, because the induced apoptosis is tumor specific, marked and adverse side effects such as inappropriate vasodilation or shock may be minimized. Preferably, the composition or method may be designed to allow sensitization of the cell or tumor to the chemotherapeutic or radiation therapy by administering at least a portion of the Smac mimetic prior to chemotherapeutic or radiation therapy. The radiation therapy, and/or inclusion of chemotherapeutic agents, may be included as part of the therapeutic regimen to further potentiate the tumor cell killing by the Smac mimetic.

Combination Therapy A combination of a Smac mimetic and a chemotherapeutic/anti-neoplastic agent and/or radiation therapy of any type may be used in embodiments of the invention and may provide a more effective approach to destroying tumor cells. Smac mimetics generally interact with IAPs, such as XIAP, cIAP-1, cIAP-2, ML-IAP, etc., and block the IAP mediated inhibition of apoptosis while chemotherapeutics/anti neoplastic agents and/or radiation therapy kills actively dividing cells by activating the intrinsic apoptotic pathway leading to apoptosis and cell death. As is described in more detail below, embodiments of the invention provide combinations of a Smac mimetic and chemotherapeutic/anti-neoplastic agents and/or radiation that may provide synergistic action against unwanted cell proliferation. This synergistic action between a Smac mimetic and a chemotherapeutic/anti-neoplastic agent and/or radiation therapy can improve the efficiency of the chemotherapeutic/anti-neoplastic agent and/or radiation therapy. This may allow for an increase in the effectiveness of current chemotherapeutic/anti-neoplastic agents or radiation treatment allowing the dose of the chemotherapeutic/anti-neoplastic agent to be lowered, thereby providing both a more effective dosing schedule, as well as a more tolerable dose of chemotherapeutic/anti-neoplastic agent and/or radiation therapy.

Additional chemotherapeutic agents Suitable chemotherapeutic agents include, but are not limited to the chemotherapeutic agents described in “Modern Pharmacology with Clinical Applications,” Sixth Edition, Craig & Stitzel, Chpt. 56, pgs. 639-656 (2004), hereby incorporated by reference. This reference describes chemotherapeutic drugs including alkylating agents, antimetabolites, anti-tumor antibiotics, plant-derived products such as taxanes, enzymes, hormonal agents such as glucocorticoids, miscellaneous agents such as cisplatin, monoclonal antibodies, immunomodulating agents such as interferons, and cellular growth factors. Other suitable classifications for chemotherapeutic agents include mitotic inhibitors and nonsteroidal anti-estrogenic analogs. Other suitable chemotherapeutic agents include toposiomerase I and II inhibitors and kinase inhibitors.

Specific examples of suitable biological and chemotherapeutic agents include, but are not limited to, cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, TRAIL, interferon (in both its alpha and beta forms), thalidomide, and melphalan. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim, and sargramostim. Chemotherapeutic compositions also comprise other members, i.e., other than TRAIL of the TNF superfamily of compounds.

For example, in one embodiment of the invention, the therapeutic compounds of the present invention may be administered with TRAIL or other chemical or biological agents which bind to and activate the TRAIL receptor(s). Many cancer cell types are sensitive to TRAIL-induced apoptosis, while most normal cells appear to be resistant to TRAIL-induced apoptosis. TRAIL-resistant cells may arise by a variety of different mechanisms including loss of the receptor, presence of decoy receptors, or overexpression of FLIP which competes for zymogen caspase-8 binding during DISC formation. Smac mimetics appear to increase tumor cell sensitivity to TRAIL leading to enhanced apoptosis, the clinical correlations of which are expected to be increased apoptotic activity in TRAIL resistant tumors, improved clinical response, increased response duration, and ultimately, enhanced patient survival rate. In support of this, reduction in XIAP levels by in vitro antisense treatment has been shown to cause sensitization of resistant melanoma cells and renal carcinoma cells to TRAIL (Chawla-Sarkar, et al., 2004). The Smac mimetics disclosed herein may bind to IAPs and inhibit their interaction with caspases, therein potentiating TRAIL-induced apoptosis.

Another embodiment of the invention provides Smac mimetics that act synergistically with a topoismerase inhibitor to potentiate their apoptotic inducing effect. Topoisomerase inhibitors inhibit DNA replication and promote DNA damage by inhibiting the enzymes that are required in the DNA repair process thereby promoting apoptosis. Therefore, export of Smac from the mitochondria into the cell cytosol is provoked by the DNA damage caused by topoisomerase inhibitors. Topoisomerase inhibitors, such as those of the Type I class, including camptothecin, topotecan, SN-38 (irinotecan active metabolite), and those of the Type II class including etoposide, show potent synergy with the Smac mimetics of the invention in a multi-resistant glioblastoma cell line (T98G), breast cancer line (MDA-MB-231), and ovarian cancer line (OVCAR-3) among others. Exemplary topoisomerase inhibiting agents that may be used in embodiments of the invention include, but are not limited to irinotecan, topotecan, etoposide, amsacrine, exatecan, gimatecan, aclacinomycin A, camptothecin, daunorubicin, doxorubicin, ellipticine, epirubicin, and mitaxantrone.

In still another embodiment of the invention, a platinum containing compound may be used as chemotherapeutic/anti-neoplastic agent in combination with a Smac mimetic. Exemplary platinum containing compounds that may synergize with a Smac mimetic include, but are not limited to, cisplatin, carboplatin, and oxaliplatin.

In yet another embodiment of the invention, taxanes may be used as the chemotherapeutic/anti-neoplastic agent that synergizes with a compound according to the invention. Taxanes may act as, for example, anti-mitotic, mitotic inhibitors or microtubule polymerization agents and include, but are not limited to, docetaxel and paclitaxel. Taxanes are characterized as compounds that promote assembly of microtubules by inhibiting tubulin depolymerization, thereby blocking cell cycle progression. Microtubules are highly dynamic cellular polymers made of alpha-beta-tubulin and associated proteins that play key roles during mitosis by participating in the organization and function of the spindle, assuring the integrity of the segregated DNA. Therefore, microtubules represent an effective target for cancer therapy, and taxanes may effectively attack this target by causing, for example, centrosomal impairment, induction of abnormal spindles, and suppression of spindle microtubule dynamics.

Another class of agents that may be utilized in embodiments of the invention includes microtubule poisons which, in contrast to taxanes, inhibit tubulin polymerization. These compounds include, but are not limited to vinca alkaloids, colchicine, and cryptophycines.

In a further embodiment, any agent that activates the intrinsic apoptotic pathway and/or causes the release of Smac or cytochrome c from the mitochondria has the potential to act synergistically with a Smac mimetic and may be used in combination with the compounds of embodiments of the invention.

Radiotherapy protocols Additionally, in several embodiments of the invention, Smac mimetic therapy may be used in connection with chemo-radiation or other radiation treatment protocols used to inhibit tumor cell growth.

Radiation therapy (or radiotherapy) is the medical use of ionizing radiation as part of cancer treatment to control malignant cells and is suitable for use in embodiments of the present invention. Although radiotherapy is often used as part of curative, primary, therapy, it is occasionally used as a palliative treatment where cure is not possible and the aim is for symptomatic relief. Radiotherapy is commonly used for the treatment of tumors, and it is common to combine radiotherapy with surgery and/or chemotherapy. The most common tumors treated with radiotherapy are breast cancer, prostate cancer, rectal cancer, head and neck cancers, gynecological tumors, bladder cancer, and lymphoma. Radiation therapy is commonly applied just to the localized area involved with the tumor. Often the radiation fields include the draining lymph nodes. It is possible, but uncommon, to give radiotherapy to the whole body or entire skin surface. Radiation therapy is usually given daily for up to 35-38 fractions (a daily dose is a fraction). These small frequent doses allow healthy cells time to grow back, repairing damage inflicted by the radiation. Three main divisions of radiotherapy are external beam radiotherapy, or teletherapy, brachytherapy or sealed source radiotherapy and unsealed source radiotherapy, which are all suitable examples of treatment protocol in the present invention. The differences relate to the position of the radiation source: external is outside the body, while sealed and unsealed source radiotherapy has radioactive material delivered internally. Brachytherapy sealed sources are usually extracted later, while unsealed sources are injected into the body. Administration of a Smac mimetic may occur prior to and/or concurrently with the treatment protocol.

### Example

Annexin V/Propidium Iodide Staining Annexin V-fluorescein isothiocyanate staining shows the ability of dimeric Smac mimetics to induce apoptosis. Cells are briefly exposed to various concentrations of dimeric Smac mimetics for 18-24 hours and removed from the assay plate by trypsinization. Cells are pelleted and resuspended in assay buffer (supplied by manufacturer). Annexin V and propidium iodide are added to the cell preparations and incubated for 1 hour in the dark at room temperature. Following the incubation, additional buffer (200 μl) is added to each tube, and the samples are analyzed by flow cytometry. In the presence of Smac mimetics apoptosis is strongly promoted as assessed by Annexin V/PI staining and analyzed by flow cytometry. The amplification in the number of apoptotic cells by IAP antagonists as compared to control was dose dependent and due to the induction of apoptosis and not via increasing the proportion of necrotic cells.

Biological and chemotherapeutics/anti-neoplastic agents and radiation induce apoptosis by activating the extrinsic or intrinsic apoptotic pathways. Since Smac mimetics relieve inhibitors of apoptotic proteins (IAPs) and thus, remove the block in apoptosis, the combination of chemotherapeutics/anti-neoplastic agents and radiation with Smac mimetics should work synergistically to facilitate apoptosis.

The relevance of this potent synergy is that it makes possible the use of the dimeric Smac mimetics, which are IAP antagonists, to improve the efficacy of conventional chemotherapeutic agents, such as, marketed platinum containing compounds (cisplatin and carboplatin). This may be accomplished by lowering the required dose of the poorly tolerated platinum containing compounds and/or by improving the response rate at the marketed dose.

The present invention is not limited to the embodiments described and exemplified above, but is capable of variation and modification within the scope of the appended claims.

1. A compound comprising a homodimer or heterodimer having monomeric units of formula (I): 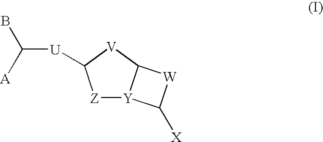
wherein:
each A is, independently, NR₁R₂, or N⁻R₁R₂R₃;
each R₁, R₂, and R₃ is, independently, hydrogen or optionally substituted alkyl, alkenyl, or alkynyl, wherein one or more carbons are optionally replaced by C═O, C═S, or a heteroatom selected from O, S, and N and wherein one or more hydrogens are optionally replaced by fluorine, a branched or unbranched alkyl or cycloalkyl, an optionally substituted aryl, arylalkyl, heteroaryl, heteroarylalkyl, OR₄, SR₄, or NR₄R₅;
each R₄ and R₅ are, independently, hydrogen or optionally substituted alkyl, alkenyl, or alkynyl, wherein one or more carbons are optionally replaced by a heteroatom selected from O, S, and N, or optionally substituted aryl, arylalkyl, heteroaryl, or heteroarylalkyl; or any two of R₁, R₂, and R₃ taken together with the nitrogen to which they are attached to form a heterocyclic group, in which one or more carbon atoms are optionally replaced by C═O, C═S, or a heteroatom selected from O, S, and N, with the proviso that the heteroatom is separated from the nitrogen atom by at least two carbons;
each B is, independently, optionally substituted alkyl, alkenyl, or alkynyl, wherein one or more hydrogens are optionally replaced by fluorine;
each U is, independently, CONH, C(O)O, C(S)O, C(S)NH, C(NH)NH, (CH₂)₁₋₅, or the inverse, thereof, wherein one or more carbons are optionally replaced by a heteroatom selected from O, S, and N;
each V and W is, independently, (CH₂)₁₋₅, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;
each X is, independently, optionally substituted alkyl, alkenyl, alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;
each Y is, independently, CH or N;
each Z is, independently, CH₂, C═O, C═S, CHSR, CHOR, or CHNR; and
each R is, independently, hydrogen or optionally substituted alkyl, alkenyl, or alkynyl; or
a pharmaceutically acceptable salt thereof

2. The compound of claim 1, having the formula (II): 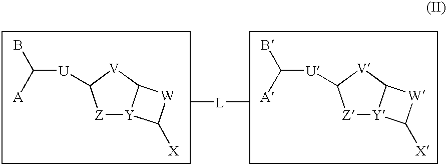
wherein:
A and A′ are each, independently, NR₁R₂ or N⁺R₁R₂R₃;
R₁, R₂, and R₃ are each, independently, hydrogen or optionally substituted alkyl, alkenyl, or alkynyl, wherein one or more carbons are optionally replaced by C═O, C═S, or a heteroatom selected from O, S, and N, and one or more hydrogens are optionally replaced by fluorine, a branched or unbranched alkyl or cycloalkyl, an optionally substituted aryl, arylalkyl, heteroaryl, heteroarylalkyl, OR₄, SR₄, or NR₄R₅;
R₄ and R₅ are each, independently, hydrogen or optionally substituted alkyl, alkenyl, or alkynyl, in which one or more carbons are optionally replaced by a heteroatom selected from O, S, and N, or optionally substituted aryl, arylalkyl, heteroaryl, or heteroarylalkyl; or any two of R₁, R₂, and R₃ taken together with the nitrogen to which they are attached to form a heterocyclic group, wherein one or more carbon atoms are optionally replaced by C═O, C═S, or a heteroatom selected from O, S, and N, with the proviso that the heteroatom is separated from the nitrogen atom by at least two carbons;
B and B′ are each, independently, optionally substituted alkyl, alkenyl, or alkynyl, wherein one or more hydrogens are optionally replaced by fluorine;
U and U′ are each, independently, CONH, C(O)O, C(S)O, C(S)NH, C(NH)NH, (CH₂)₁₋₅, or the inverse wherein one or more carbons are optionally replaced by a heteroatom selected from O, S, and N;
V, V′, W and W′ are each, independently, (CH₂)₁₋₅, wherein one or more carbons are optionally replaced by C═O, C═S, or a heteroatom selected from O, S, and N, and one or more hydrogens in CH₂ groups are optionally replaced by a branched or unbranched alkyl or cycloalkyl, an optionally substituted aryl, arylalkyl, heteroaryl, heteroarylalkyl, OR₄, SR₄, or NR₄R₅;
X and X′ are each, independently, optionally substituted C₁₋₁₈-alkyl, C₂₋₁₈-alkenyl, or C₂₋₁₈-alkynyl, aryl, or heteroaryl, wherein one or more carbons are optionally replaced by C═O, C═S, or a heteroatom selected from O, S, and N, and one or more hydrogens are optionally replaced by a branched or unbranched alkyl or cycloalkyl, an optionally substituted aryl, arylalkyl, heteroaryl, heteroarylalkyl, OR₄, SR₄, or NR₄R₅;
Y and Y′ are each, independently, CH or N;
Z and Z′ are each, independently, CH₂, C═O, C═S, CHSR, CHOR, or CHNR;
each R is, independently, hydrogen, or optionally substituted alkyl, alkenyl, or alkynyl; and
L is one or more bonds or one or more linkers covalently linking one or more of positions V, W, and X, with V′, W′, and X′; or
a pharmaceutically acceptable salt thereof

3. The compound of claim 2, wherein the L covalently links two identical monomeric units or L covalently links two non-identical monomeric units.

4. The compound of claim 2, wherein L is selected from alkylene, alkenylene, alkynylene, cycloalkylene, cycloalkylalkylene, aryl, arylalkylene, arylalkylalkylene, and heterocycloalkylene, heterocycloalkylalkylene, heteroaryl and heteroarylalkylene where one or more carbon atoms are optionally replaced with N, O, or S, optionally-substituted alkylene, alkenylene, alkynylene cycloalkylene, cycloalkylalkylene, heterocycloalkylene, heterocycloalkylalkylene, aryl, arylalkylene, arylalkylalkylene, and heteroaryl and heteroarylalkylene where one or more carbon atoms are optionally replaced with N, O, or S, amino, substituted amino, oxygen atom, sulfide, sulfoxide, sulfone and disulfide.

5. The compound of claim 2, wherein L is selected from —CH₂CH₂—, —CH₂CH₂CH₂—, —CH═CH—, 1,4-phenyl, 2,5-thiophenyl, —CH(OH)CH(OH)—, —CH₂CH—O—CHCH₂—, and —CH₂C≡CC≡H₂—.

6. The compound of claim 2, having a formula selected from a compound of formula (III): 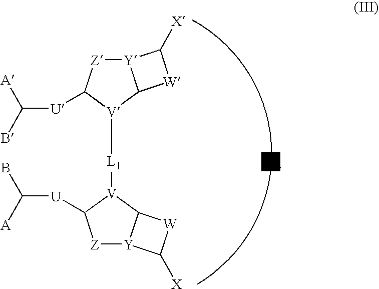
wherein:
A, A′, B, B′, U, U′, Z, Z′, Y, Y′, V, V′, W, W′, X, and X′ are defined as in claim 2; and
L₁ and L₂, independently, link position V with V′ and X with X′; or
a pharmaceutically acceptable salt thereof;
a compound of general formula (IV): 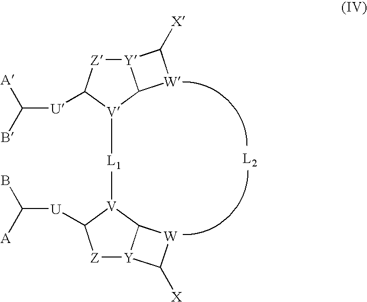
wherein:
A, A′, B, B′, U, U′, Z, Z′, Y, Y′, V, V′, W, W′, X, and X′ are defined as in claim 2; and
L₁ and L₂, independently, link position V with V′ and W with W′; or
a pharmaceutically acceptable salt thereof;
a compound of general formula (V): 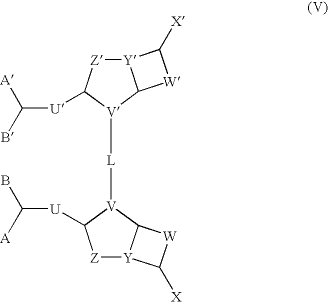
wherein:
A, A′, B, B′, U, U′, Z, Z′, Y, Y′, V, V′, W, W′, X, and X′ are defined as in claim 2; and
L links position V with V′; or
a pharmaceutically acceptable salt thereof;
a compound of general formula (VI): 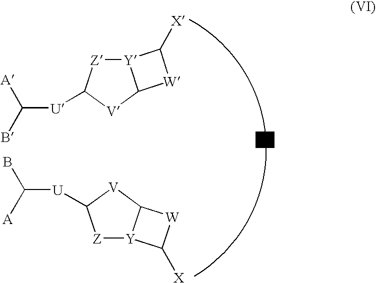
wherein:
A, A′, B, B′, U, U′, Z, Z′, Y, Y′, V, V′, W, W′, X, and X′ are defined as in claim 2; and
L links position X with X′; or
a pharmaceutically acceptable salt thereof; and
a compound of general formula (VII): 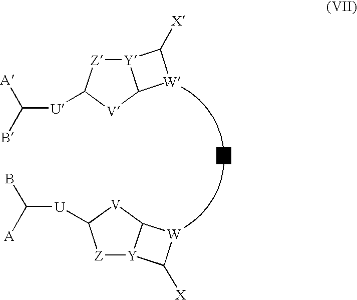
wherein:
A, A′, B, B′, U, U′, Z, Z′, Y, Y′, V, V′, W, W′, X, and X′ are defined as in claim 2; and
L links position W with W′; or
a pharmaceutically acceptable salt thereof

7. A compound comprising a homodimer or heterodimer having monomeric units of formula (VIII): 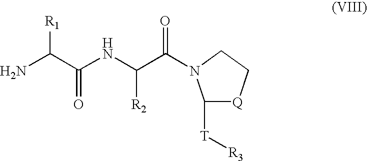
wherein:
each R₁ is, independently, alkyl or haloalkyl;
each R₂ is, independently, branched or unbranched alkyl or cycloalkyl, or substituted or unsubstituted aryl, arylalkyl, heteroaryl, or heteroarylalkyl;
each R₃ is, independently, branched or unbranched alkyl or cycloalkyl, or substituted or unsubstituted aryl, arylalkyl, heteroaryl, or heteroarylalkyl;
each S is, independently, (CH₂)₀₋₃ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and
each T is, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is a branched or unbranched alkyl, cycloalkyl or substituted or unsubstituted aryl, arylalkyl, heteroaryl, or heteroarylalkyl teroaryl; or
a pharmaceutically acceptable salt thereof

8. The compound of claim 7, having the formula (IX): 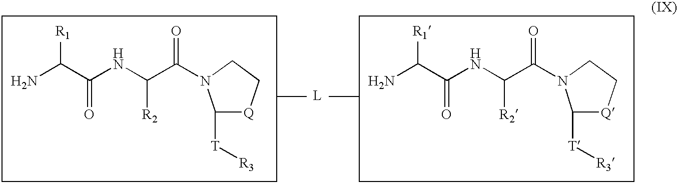
wherein:
R₁ and R₁′ are each, independently, alkyl or haloalkyl;
R₂ and R₂′ are each, independently, branched or unbranched alkyl or cycloalkyl, or substituted or unsubstituted aryl, arylalkyl, heteroaryl, or heteroarylalkyl;
R₃ and R₃′ are each, independently, branched or unbranched alkyl or cycloalkyl, or substituted or unsubstituted aryl, arylalkyl, heteroaryl, or heteroarylalkyl;
Q and Q′ are each, independently, (CH₂)₀₋₃ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and
T and T′ are each, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃(CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and
L is one or more linkers covalently linking one or more of the positions R₂, R₃, Y, with R₂′, R₃′, Y′; and
pharmaceutically acceptable salts thereof.

9. The compound of claim 8, wherein L covalently links two identical monomeric units or L covalently links two non-identical monomeric units.

10. The compound of claim 8, wherein L is selected from alkylene, alkenylene, alkynylene, cycloalkylene, cycloalkylalkylene, aryl, arylalkylene, arylalkylalkylene, and heterocycloalkylene, heterocycloalkylalkylene, heteroaryl and heteroarylalkylene where one or more carbon atoms are optionally replaced with N, O, or S, optionally-substituted alkylene, alkenylene, alkynylene cycloalkylene, cycloalkylalkylene, heterocycloalkylene, heterocycloalkylalkylene, aryl, arylalkylene, arylalkylalkylene, and heteroaryl and heteroarylalkylene where one or more carbon atoms are optionally replaced with N, O, or S, amino, substituted amino, oxygen atom, sulfide, sulfoxide, sulfone and disulfide.

11. The compound of claim 8, wherein L is selected from —CH₂CH₂—, —CH₂CH₂CH₂—, —CH═CH—, 1,4-phenyl, 2,5-thiophenyl, —CH(OH)CH(OH)—, —CH₂CH—O—CHCH₂—, and —CH₂C≡CC≡H₂—.

12. The compound of claim 8, having a formula selected from a compound of formula (X): 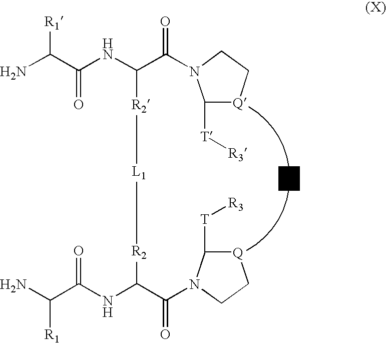
wherein:
R₁, R₁′, R₂, R₂′, R₃, R₃′, Q, Q′, T, and T′ are defined as in claim 11; and
L₁ and L₂, independently, link position R₂ with R₂′ and Q with Q′; or
a pharmaceutically acceptable salt thereof;
a compound of general formula (XI): 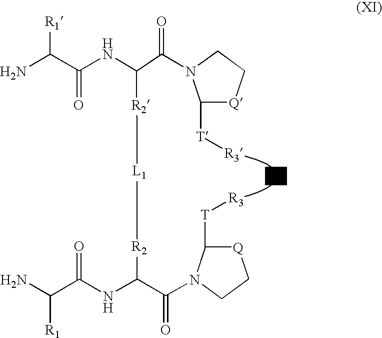
wherein:
R₁, R₁′, R₂, R₂′, R₃, R₃′, Q, Q′, T, and T′ are defined as in claim 11; and
L₁ and L₂, independently, link position R₂ with R₂′ and R₃ with R₃′; or
a pharmaceutically acceptable salt thereof;
a compound of general formula (XII): 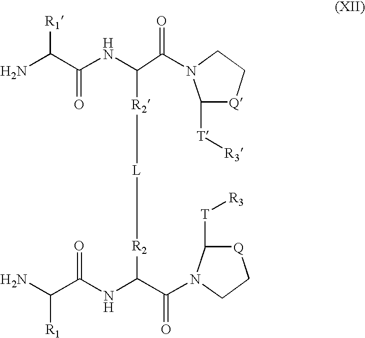
wherein:
R₁, R₁′, R₂, R₂′, R₃, R₃′, Q, Q′, T, and T′ are defined as in claim 11; and
L covalently links position R₂ with R₂′; or
a pharmaceutically acceptable salt thereof;
a compound of general formula (XIII): 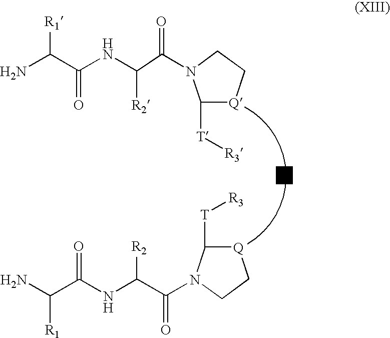
wherein:
R₁, R₁′, R₂, R₂′, R₃, R₃′, Q, Q′, Z, and Z′ are defined as in claim 11; and
L covalently links position Q with Q′; or
a pharmaceutically acceptable salt thereof; and
a compound of general formula (XIV): 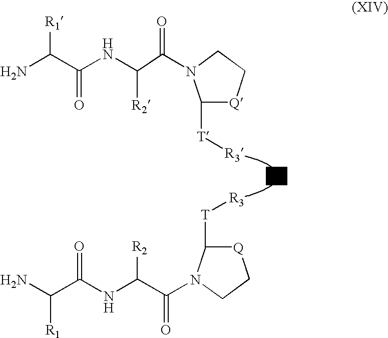
wherein:
R₁, R₁′, R₂, R₂′, R₃, R₃′, Q, Q′, T, and T′ are defined as in claim 11; and
L covalently links position R₃ with R₃′; or
a pharmaceutically acceptable salt thereof

13. A compound comprising a homodimer or heterodimer having monomeric units of general formula (XV): 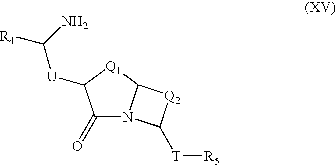
wherein:
each R₄ is, independently, alkyl or haloalkyl;
each R₅ is, independently, branched or unbranched alkyl or cycloalkyl, or substituted or unsubstituted aryl, arylalkyl, heteroaryl, or heteroarylalkyl;
each U is, independently, CONH, CH₂O, CH₂NH, CH₂S, or (CH₂)₁₋₃;
each Q₁ is, independently, (CH₂)₁₋₅ wherein one or more carbons can be replaced by one or more heteroatoms selected from O, S, and N; and one or more hydrogens in CH₂ groups can be replaced by a branched or unbranched alkyl or cycloalkyl, or substituted or unsubstituted aryl, arylalkyl, heteroaryl, or heteroarylalkyl;
each Q₂ is, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and
each T is, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; or
a pharmaceutically acceptable salt thereof.

14. The compound of claim 1, having the formula (XVI): 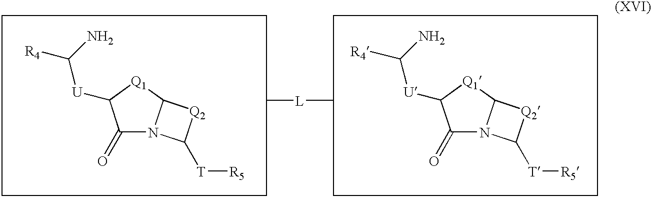
wherein:
R₄ and R₄′ are each, independently, alkyl or haloalkyl;
R₅ and R₅′ are each, independently, branched or unbranched alkyl or cycloalkyl, or substituted or unsubstituted aryl, arylalkyl, heteroaryl, or heteroarylalkyl;
U and U′ are each, independently, CONH, CH₂O, CH₂NH, CH₂S, or (CH₂)₁₋₃;
Q₁ and Q₁′ are each, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and
Q₂ and Q₂′ are each, independently, (CH₂)₁₋₅ wherein one or more carbons are optionally replaced by one or more heteroatoms selected from O, S, and N; and one or more hydrogens are optionally replaced by a branched or unbranched alkyl or cycloalkyl, or substituted or unsubstituted aryl, arylalkyl, heteroaryl, or heteroarylalkyl; and
T and T′ are each, independently, CONH, CH₂O, NHCO, (CH₂)₁₋₄, (CH₂)₁₋₃CONH(CH₂)₀₋₃, (CH₂)₁₋₃S(CH₂)₀₋₃, (CH₂)₁₋₃NH(CH₂)₀₋₃, (CH₂)₁₋₃NHCO(CH₂)₀₋₃, (CH₂)₁₋₃NHSO₂(CH₂)₀₋₃, (CH₂)₁₋₃NHC(O)NH(CH₂)₀₋₃, (CH₂)₁₋₃NHC(S)NH(CH₂)₀₋₃, or (CH₂)₁₋₃NR′(CH₂)₀₋₃, wherein R′ is branched or unbranched alkyl or cycloalkyl, or substituted or unsubstituted aryl, arylalkyl, heteroaryl, or heteroarylalkyl; and
L is one or more linkers covalently linking one or more of the positions Y₁, Y₂, R₂, with Y₁′, Y₂′, R₂′; or
a pharmaceutically acceptable salt thereof

15. The compound of claim 14, wherein L covalently links two identical monomeric units or L covalently links two non-identical monomeric units.

16. The compound of claim 14, wherein L is selected from alkylene, alkenylene, alkynylene, cycloalkylene, cycloalkylalkylene, aryl, arylalkylene, arylalkylalkylene, and heterocycloalkylene, heterocycloalkylalkylene, heteroaryl and heteroarylalkylene where one or more carbon atoms are optionally replaced with N, O, or S, optionally-substituted alkylene, alkenylene, alkynylene cycloalkylene, cycloalkylalkylene, heterocycloalkylene, heterocycloalkylalkylene, aryl, arylalkylene, arylalkylalkylene, heteroaryl, and heteroarylalkylene where one or more carbon atoms are, optionally, replaced with N, O, or S, amino, substituted amino, oxygen atom, sulfide, sulfoxide, sulfone, and disulfide.

17. The compound of claim 14, wherein L is selected from —CH₂CH₂—, —CH₂CH₂CH₂—, —CH═CH—, 1,4-phenyl, 2,5-thiophenyl, —CH(OH)CH(OH)—, —CH₂CH—O—CHCH₂—, and —CH₂C≡CC≡H₂—.

18. The compound of claim 14, having a formula selected from a compound of formula (XVII): 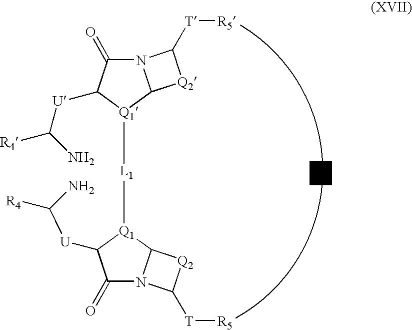
wherein:
R₄, R₄′, U, U′, Q₁, Q₁′, Q₂, Q₂′, T, T′, R₅, and R₅′ are defined as in claim 14; and
L₁ and L₂, independently, link position Q₁ with Q₁′ and R₅ with R₅′; or
a pharmaceutically acceptable salt thereof;
a compound of general formula (XVIII): 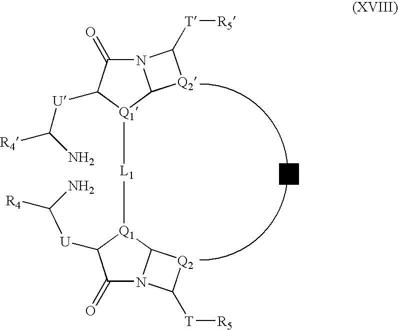
wherein:
R₄, R₄′, U, U′, Q₁, Q₁′, Q₂, Q₂′, T, T′, R₅, and R₅′ are defined as in claim 14; and
L₁ and L₂, independently, link position Q₁ with Q₁′ and Q₂ with Q₂′; or
a pharmaceutically acceptable salt thereof;
a compound of general formula (XIX): 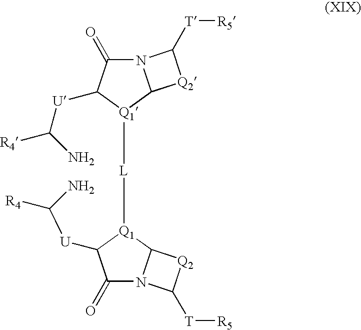
wherein:
R₄, R₄′, U, U′, Q₁, Q₁′, Q₂, Q₂′, T, T′, R₅, and R₅′ are defined as in claim 14; and
L covalently links position Q₁ with Q₁′; or
a pharmaceutically acceptable salt thereof;
a compound of general formula (XX): 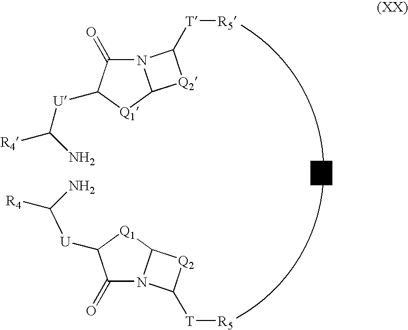
wherein:
R₄, R₅′, U, U′, Q₁, Q₁′, Q₂, Q₂′, T, T′, R₅, and R₅′ are defined as in claim 14; and
L covalently links position R₅ with R₅′; or
a pharmaceutically acceptable salt thereof; and
a compound of general formula (XXI): 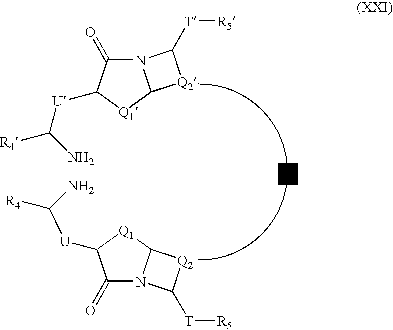
wherein:
R₄, R₄′, U, U′, Q₁, Q₁′, Q₂, Q₂′, T, T′, R₅ and R₅′ are defined as in claim 14; and
L covalently links position Q₂ with Q₂′; or
a pharmaceutically acceptable salt thereof.

19. A pharmaceutical composition comprising a compound of claim 1 and a pharmaceutically acceptable excipient.

20. The composition of claim 19, further comprising a second therapeutic agent selected from chemotherapeutic agents, radiation, immunotherapeutic agents, photodynamic therapy agents, and combinations thereof.
